(19) United States
(12) Patent Application Publication
(10) Pub. No.: US 2002/0068754 A1
(43) Pub. Date: Jun. 6, 2002

(54) Combination of adrenergic agonist and aryl-cyclo-alkanolamine for relieving chronic pain without adverse side effects

(76) Inventors: John W. Olney, Ladue, MO (US); Nuri B. Farber, University City, MO (US); Vesna Jevtovic-Todorovic, Olivette, MO (US)

Correspondence Address: Patrick D. Kelly, 11939 Manchester #403, St. Louis, MO, 63131, US

(21) Appl. No.: 09/820,080

(22) Filed: Mar. 28, 2001

Related U.S. Application Data
(63) Continuation-in-part of application No. 09/536,888, filed on Mar. 28, 2000.
Application No. 09/536,888 is a continuation-in-part of application No. 09/030,688, filed on Feb. 25, 1998, now abandoned.

(51) Int. Cl. A61K 31/445; A61K 31/40; A61K 31/155; A61K 31/137

(52) U.S. Cl. 514/327; 514/408; 514/634; 514/649

(57) ABSTRACT

This invention discloses that a combination of two drugs, from two different and unrelated categories, provides effective and long-lasting relief from neuropathic pain. Both drugs can be taken orally, in a convenient, painless, non-invasive manner that does not require injections. One drug is an aryl-cyclo-alkanolamine (ACAA) which has antagonist (receptor-blocking) activity at the NMDA subclass of glutamate receptors; such drugs include procyclidine, biperiden, and trihexyphenidyl. The other drug is an α2 adrenergic agonist, exemplified by clonidine. When an ACAA drug which blocks NMDA receptors, such as procyclidine, is administered together with an α2 adrenergic agonist such as clonidine, these drugs mutually potentiate one another's neuropathic pain-relieving action, and provide potent and sustained neuropathic pain relief, even when each agent is administered at a low dosage that is below its threshold for causing adverse side effects.

### RELATED APPLICATIONS

This application is a continuation-in-part of patent application Ser. No. 09/536,888, filed on Mar. 28, 2000, which in turn was a continuation-in-part of application Ser. No. 09/030,688, filed on Feb. 25, 1998, now abandoned.

### GOVERNMENT SUPPORT

The research which led to this invention was supported in part by a grant from the National Institutes of Health. Accordingly, the federal government has certain rights in this invention.

### BACKGROUND OF THE INVENTION

The present invention relates to neurology and pharmacology, and to drugs which can treat and control various types of chronic pain (including neuropathic pain) without causing adverse side effects.

Available drug treatments for chronic and severe pain are subject to various limitations and shortcomings. Drugs such as ibuprofen, naproxen, acetaminophen, and aspirin do not have nearly the same potency as opiates, such as morphine. Opiates are powerful, but they pose high risks of dependence and addiction, and they cause serious side effects, including drowsiness, gastrointestinal disorders, sexual dysfunctions, etc.

In addition, opiates are not effective in treating certain types of pain, the most notable example being “neuropathic pain”, a type of pain experienced when a patient's nervous system is suffering from some type of pathological damage or condition (hence the term “neuro-pathic”). There are several different types of neuropathic pain, but a common denominator of all types is that they do not respond adequately to opiate medications; this trait is so prevalent that physicians tend to view “neuropathic pain” as being synonymous with pain that does not respond adequately to opiates. When a pain condition in a specific patient will not respond adequately to opiates, it is assumed that: (i) the patient is suffering from neuropathic pain; and, (ii) the neuronal pathways that are causing or aggravating the pain condition in that patient are not pathways that use opiate receptors; rather, they convey pain messages via other neurotransmitter/receptor systems.

Most cases of neuropathic pain appear to involve chronic conditions that arise when nerve fibers or endings in a certain part of the body (or the larger neuronal networks they are connected to, which may include neurons located in the spinal cord) have become hyper-sensitive (also referred to as being hyper-irritable, or being in a “kindled” or “wind-up” condition). In this condition, certain neuronal endings, receptors, or other components or circuits are in a chronic state of abnormally high sensitivity, and/or have abnormally low triggering thresholds. In this state, they convey (either spontaneously, or in response to very mild stimuli that would not be painful to a healthy person) far too many nerve signals of the type classified by neurologists as “nociceptive” nerve impulses (i.e., nerve impulses interpreted by the nervous system as signalling pain; this class of signals is distinct from other types of nerve impulses, such as sensory signals for light, smell, taste, etc.).

A person with a hypersensitized or “kindled” neuropathic condition is comparable to a person whose eyes were dilated by a drug, during an eye exam, and who is then forced to look directly into a bright light. Just as bright light is very unpleasant and even painful to a person with dilated eyes, a patient subject to neuropathic pain will suffer from what appear to be artificially amplified surges and waves of nociceptive nerve signals, from the affected part(s) of the body. Those amplified signals will be perceived as pain, even when no stimulus has been inflicted that would be perceived as painful by a person who is not suffering from the neuropathic hypersensitivity.

Well-known examples of neuropathic pain include: (i) chronic painful states that occur in association with diabetes, often referred to as “diabetic neuropathy”; (ii) chronic pain associated with traumatic injury to the peripheral nervous system; (iii) chronic pain resulting from herpes zoster (also known as shingles, or post-herpetic neuropathy) or similar infections that attack and damage nerve fibers or endings; (iv) post-operative pain, which arises after surgery and then lingers far beyond a normal convalescent period; (v) “phantom limb” pain, in which an amputee suffers from feelings of pain or discomfort that seems to originate in the missing limb; and (vi) causalgia, which refers to pain that is perceived as a burning sensation (“causalgia” comes from the same Greek root as “cautery”, and has nothing to do with causation).

In addition, certain patterns of neuropathic pain commonly arise in certain parts of the central or peripheral nervous system. Examples include trigeminal pain, which afflicts the trigeminal nerve in the facial region, and arachnoiditis, in which a certain layer of membrane that generally surrounds the brain and spinal cord becomes damaged and/or inflamed, usually due to trauma, surgery, or infection.

All of these conditions appear to share at least some underlying mechanisms and/or traits. In particular, they share the common distinction that they all involve chronic pain, and the pain reaches a level regarded by the patient and the treating physician as “intractable”. As used herein, the term intractable implies that: (i) a pain condition cannot be adequately relieved by opiates or other currently available pain medications; and (ii) any short-term relief provided by opiates or other drugs is usually accompanied by serious side effects, such as sedation, gastrointestinal disorders, etc.

Any references herein to relief, short-term relief, or similar terms, refer to an effective form of non-sedating relief. Obviously, if a patient is rendered unconscious by a powerful sedative, the patient will feel no pain for as long as he or she remains unconscious. However, just as obviously, that is not the goal of an effective treatment for chronic pain.

In this context, terms such as sedative, sedating, or sedation do not imply a treatment that is soothing, relaxing, and appreciated; instead, if a drug must be used on a chronic, daily basis for months or years, “sedating” indicates a highly undesired and often debilitating interference with mental clarity, and should be regarded in the same general category as “addictive narcotic”. One of the most troubling and depressing side effects of pain-killing drugs, in the view of anyone who must take them to treat chronic intractable pain, is the severe disruption they impose on a patient's mental clarity. Sedating pain-killers make it tiring and difficult, and often impossible, for a patient to have the sort of normal, healthy, happy activities and relationships that he or she enjoyed before the chronic pain began. Sedation also substantially increases the risk of serious accidents, such as a collision while driving, or a fall while walking (which poses a serious risk of a broken hip, leg, or arm, especially in elderly patients).

If an effective and non-sedating treatment for neuropathic or other chronic or intractable pain could be discovered and made publicly available, it would be a major advance over opiates and other currently available drug treatments, and it would offer blessed relief to literally millions of people around the world.

This invention discloses what appears to be a major advance in that direction. To understand how this drug treatment works, and which drugs are covered by certain terms used in the text and claims below, additional background information needs to be provided on certain types of “receptor” systems that are present on the surfaces of neurons.

Neurotransmitter Systems

Several systems, involving neurotransmitter molecules and the neuronal receptors they interact with, are involved in transmitting nerve signals (including pain signals) from one neuron to another.

Mammalian brains are extraordinarily complex, and the background section of a patent is not the proper place for a detailed analysis of how neurons transmit nerve signals to each other. Nevertheless, several concepts and terms need to be raised and discussed, since they are relevant to this invention. Accordingly, the following is a brief overview of several relevant components and processes. For more information, a good introduction to the structure and functioning of neurons can be found in physiology textbooks, such as Guyton and Hall, Textbook of Medical Physiology, W. B. Saunders, 9th edition, 1996). For more information, a single-volume textbook widely used in medical schools is Principles of Neuroscience (E. R. Kandel et al, editors, 4th ed., McGraw Hill, 2000). For still more information, the most widely-used treatise on the nervous system is the Encyclopedia of Neuroscience (first edition, edited by Adelman, 1987, or second edition, edited by Adelman and Smith, 1999).

Very briefly, “neurotransmitter receptors” are specialized membrane sites that are exposed and accessible on certain surface areas of neurons, in the “synaptic” junctions between neurons. Transmission of a nerve impulse (also called a signal, message, depolarizing event, etc.) from one neuron to another, through a synaptic junction, is achieved by the release of neurotransmitter molecules (such as glutamate or acetylcholine, two of the most important neurotransmitters) by the transmitting neuron, into the liquid that fills the gap in a synaptic junction with the signal-receiving neuron. The neurotransmitter molecule briefly binds to an exposed and accessible portion of a receptor protein, which straddles the neuronal membrane. This binding reaction between the transmitter molecule and the receptor protein triggers a cellular reaction (such as opening an ion channel, in glutamate receptors).

With a few exceptions, each type of neuronal receptor is triggered by only one type of neurotransmitter. Therefore, each of the various classes of transmitter-and-receptor pairings in the central nervous system (CNS) can be regarded as a “system”. The CNS has a glutamate system, an acetylcholine system (usually called the cholinergic system), a dopamine system, a serotonin system, an adrenergic system, and a variety of other similar systems using other specific neurotransmitters.

In addition, most receptor systems have multiple subclasses. For example, the glutamate receptor system is subdivided into three major subclasses, designated as the NMDA, kainate, and AMPA subclasses. In the brain, all three subclasses are triggered by glutamate (and by aspartate, to a much lesser extent). However, in cell culture tests, scientists have identified “probe drugs” which can selectively activate one particular subclass of glutamate receptors. A probe drug called N-methyl-D-aspartate (abbreviated as NMDA), which does not exist inside the brain, powerfully triggers a certain class of glutamate receptors, but has little or no effect on the other two subclasses. Accordingly, those glutamate receptors that are activated by NMDA are called NMDA receptors.

In a similar manner, acetylchcoline (ACh) receptors are subdivided into the “muscarinic” and “nicotinic” classes, and those classes are divided into still more subclasses; for example, muscarinic ACh receptors are subdivided into m1, m2, m3, m4, and m5 subclasses.

Another transmitter-receptor system that is important to this invention is the “adrenergic” system. Since it is directly relevant to this invention, it is discussed in some detail below.

The glutamate and acetylcholine (ACh) systems are both excitatory, since the action of glutamate or ACh at those receptors normally results in a neuronal firing event (unless that receptor or neuron has been inhibited by one of the inhibitory neurotransmitter systems). Various other transmitter-receptor pairings are regarded as inhibitory systems. When an inhibitory neurotransmitter reacts with an inhibitory receptor, rather than triggering a neuronal firing event in a manner comparable to glutamate or ACh, the inhibitory reaction causes the neuron to become less susceptible to excitation, even if its synaptic receptors are contacted by glutamate or ACh. The dopamine, serotonin, and GABA systems are all regarded as inhibitory.

For the most part, the adrenergic system is regarded as an inhibitory system, rather than an excitatory system. The adrenergic system is quite complicated, and acts in various different ways, both inside the CNS, and outside the CNS. Inside the CNS, it is regarded as predominantly inhibitory.

Inhibitory systems play a crucial role in the proper functioning of the CNS. By way of analogy, they are similar to a tuning system which allows a television set to process electromagnetic waves that contain dozens or even hundreds of channels, and present a single coherent picture on the screen, with accompanying sound. In a similar manner, inhibitory neurotransmitters allow the CNS to process huge numbers of input signals in ways that generate coherent thoughts, useful muscle and organ control, etc.

Another pair of terms relevant to this invention also needs to be established. As used herein, a receptor “agonist” is a molecule which increases activity levels at that type of receptor. By contrast, a receptor “antagonist” is a molecule which reduces activation of a receptor type by its normal neurotransmitter.

Many agonists exert their effects by binding directly to the receptor, in a manner which “triggers” the receptor. However, other types of agonists (as that term is used herein) use alternate methods, such as increasing the concentration of the natural transmitter molecule in the extra-cellular fluid surrounding the neuronal receptors. Examples include cholinesterase inhibitor drugs, which prevent ACh molecules from being degraded rapidly by cholinesterase enzymes, and “serotonin uptake inhibitors”, which slow down the rate at which serotonin is pumped back into neurons after it has been released.

It also should be recognized that not all agonists are excitatory. For example, a GABA agonist will inhibit neuronal firings, since that is the same function exerted in the CNS by GABA.

All references herein to agonists or antagonists (or to drugs of any sort) refer to “exogenous” compounds, i.e., compounds (other than foods) that are synthesized outside an animal or human, and then administered to the animal or human. As used herein, terms such as agonist, antagonist, or drug exclude naturally occurring neurotransmitters that are found under normal conditions inside the CNS of an animal or person. The term “compound” is used broadly herein; in addition to drugs, it includes newly-synthesized molecules that are of interest for possible medical use. If a compound passes various initial hurdles and is actually tested in animals, and is shown in such tests to be pharmaceutically acceptable, it is thereafter referred to as a drug.

Much of neurology and neuropharmacology focus on “exogenous” (non-natural) drugs that mimic the action of neurotransmitters at certain classes (or subclasses) of neuronal receptors. Research on neuronal receptor systems is often performed using agonist drugs that activate one or more classes of neuronal receptors, and/or antagonist drugs that prevent or reduce activation of one or more classes of neuronal receptors. This has been rendered possible and even routine by means of various research instruments, including very small electrodes that can detect a single firing event by a single neuron, either in vitro (using cell culture tests), or in vivo (using lab animals, or even human subjects during a neurosurgical operation).

Accordingly, neuroactive drugs are commonly described by reference to their interactions with a class of neuronal receptors. As one example, MK-801, phencyclidine, ketamine, and tiletamine are all immediately recognized among neurologists as NMDA antagonist drugs, even though their chemical structures are substantially different from each other, since they all bind to a certain receptor site associated with the NMDA receptor complex.

Glutamate and NMDA Receptors; NMDA Antagonist Drugs

Glutamate (the ionized form of glutamic acid, an amino acid) is a major excitatory neurotransmitter. Unlike molecules of acetylcholine, which are rapidly broken apart by enzymes after they are released by a synaptic receptor, glutamate molecules that have been released by a synaptic receptor must be pumped back inside a neuron or glial cell.

The glutamate pumping mechanism requires energy to drive it. Therefore, if a region of the brain or spinal cord begins to suffer from an ischemic (inadequate blood flow) or hypoxic (inadequate oxygen supply) crisis, due to a stroke, cardiac arrest, head trauma, etc., excess free glutamate molecules can begin to accumulate in that portion of the brain. Because of certain ways that stressed neurons and glial cells respond to that type of crisis, this can lead to a type of over-excitation that can permanently damage or even kill neurons. This phenomenon, known as “excitotoxicity”, can severely worsen the extent and severity of brain damage that occurs in the hours and days after a stroke, head trauma, cardiac arrest, severe epileptic seizure, or similar crisis.

By the early 1980s, it was becoming established (published studies are reviewed in Olney 1990 and Choi 1992) that excessive activation of NMDA receptors will aggravate and increase the brain damage caused by a stroke, head trauma, or similar crisis. In response, a number of pharmaceutical companies began developing NMDA antagonist drugs (i.e., drugs which suppress glutamate-induced neuronal firings by blocking the NMDA receptor complex) as neuroprotective (anti-excitotoxic) agents.

By the mid-1980's, a number of candidate NMDA antagonists were becoming available to neurology researchers. As soon as these drugs became available, neurologists (who were well aware by then of the highly important role of NMDA receptors in CNS functioning) began testing these compounds, to evaluate their effects on nearly every conceivable type of neurological functioning, ranging from pain, to sensory processing, to memory. By the mid to late 1980s, a number of researchers had reported that NMDA antagonist drugs can relieve neuropathic pain (e.g., Raigorodsky et al 1987; Aaronsen et al 1987; Woolf et al 1989; Davar et al 1991; Seltzer et al 1991; Yamamoto et al 1992; Mao et al 1992; Kristensen et al 1992; Backonja et al 1994).

However, by the late 1980's, neurologists were also realizing that NMDA antagonist drugs can cause serious side effects, including psychotic reactions and physical injury to neurons, when administered at the dosages required to relieve neuropathic pain, or to prevent excitotoxic neuronal damage after a stroke or other acute insult.

In humans, it is well documented that NMDA antagonist drugs cause psychotic side effects, including hallucinations and delusions. People who abuse phencyclidine (also known as PCP, or “angel dust”) often suffer acute psychotic episodes, and surgical patients emerging from ketamine anesthesia often suffer “emergence” psychoses unless they are also treated with a second drug to suppress that reaction. Both phencyclidine and ketamine are NMDA antagonists.

In experimental animals, analysis of brain tissue following treatment with an NMDA antagonist drug reveals that NMDA antagonists cause acute physical injury to neurons which, in extreme cases, can lead to neuronal death and permanent brain damage. These neurotoxic effects occur in several specific regions of the brain, including two regions known as the posterior cingulate and retrosplenial (PC/RS) cortex, as well as the hippocampus. These brain regions receive input from a variety of other brain regions, and they perform important “central processing” functions (which can also be regarded as “switchboard” or “clearinghouse” functions).

The damage that occurs in the most vulnerable portions of the brain is manifested, at a fairly early stage, by the formation of empty “vacuoles” inside the affected neurons. These vacuoles, which do not occur in healthy neurons, provide a useful and convenient way to measure and quantify the damage to vulnerable neurons, since the vacuoles can be easily seen and counted, using microscopic examination of tissue slices. Other types of neuronal damage can also be detected if the various analytical steps are taken; as one example, affected neurons begin expressing “heat shock” proteins, which indicate that the neurons containing those proteins are being subjected to severe and potentially lethal stress. In addition, potent NMDA antagonists at substantial dosages can cause neuronal death, which can be measured using various types of stains that cannot permeate into viable neurons. Neuronal vacuoles, heat shock protein expression, mitochondrial damage, and other neurotoxic manifestations are discussed in more detail in items such as U.S. Pat. No. 5,877,173 (Olney et al 1999).

There is considerable evidence indicating that these same regions of the brain are also involved in causing psychotic effects, in humans. Evidence that the same neural networks are involved in both the psychotic and neurotoxic changes strongly suggests that humans who take NMDA antagonist drugs, in addition to suffering transient effects such as hallucinations, are at a serious risk of the same type of physical injury to neurons that can be shown to occur in test animals.

Because NMDA antagonist drugs are known to cause psychoto-mimetic effects in humans, and are strongly suspected of posing serious and unacceptable risks of permanent brain damage, efforts to develop NMDA antagonist drugs for neuroprotective purposes following a stroke or similar crisis, or for use in treating neuropathic pain or other chronic pain, have uniformly failed. Even though at least a dozen major drug companies launched major research programs, attempting to develop effective yet safe NMDA antagonists, every such effort has failed. As this is being written, in March 2001 (nearly 20 years after the first known NMDA antagonist drugs were announced), not a single known NMDA antagonist drug has ever been approved for public sale and use, by the U.S. Food and Drug Administration.

The only publicly available drugs that are known to have some degree of NMDA antagonist activity fall into two categories. The first category includes two surgical anesthetics, ketamine and tiletamine, and these were approved by the FDA many years before their role as NMDA antagonists were known. The second category includes a few drugs (such as dextromethorphan and procyclidine) which have strong primary activities involving completely different neuronal receptor systems, and which were discovered, after many years of public use, to have relatively mild and weak secondary activities as NMDA antagonists.

The failure of any drug company to successfully launch any known NMDA antagonist drug as a commercial product is also noteworthy, and perhaps even peculiar, because it has been known for nearly 10 years that any of several types of drugs can effectively block the neurotoxic side effects of NMDA antagonists, in test animals. When used for this purpose, these drugs are referred to herein as “safener” drugs, borrowing a term that is well known in other chemical industries, such as the herbicide industry. Administration of one or more of these “safener” drugs can effectively block the neurotoxic side effects caused by NMDA antagonists, in test animals.

These types of “safener” drugs can be divided into several categories, depending on which neurotransmitter system they affect. Briefly, they include: (1) anticholinergic drugs, such as scopolamine, as described in Olney et al 1991 and U.S. Pat. No. 5,034,400 (Olney 1991); (2) GABA agonist drugs, as described in U.S. Pat. No. 5,474,990 (Olney 1995); (3) alpha-2 (α2) adrenergic agonists, discussed in detail below; and, (4) drugs that suppress activity at kainate and AMPA receptors, as described in U.S. Pat. No. 5,767,130 (Olney 1998).

Despite all these options (and several others, not mentioned above), most of which involve well-known and long-accepted drugs, and despite the demonstration in test animals that the neurotoxic side effects of potent NMDA antagonists can be greatly reduced or entirely eliminated by these types of “safener” drugs, the pharmaceutical industry has steered entirely away from any effort to develop and commercialize any drug combination involving an NMDA antagonist drug, accompanied by a second drug that would prevent the neurotoxic side effects of the NMDA antagonist drug. Instead, efforts have continued to focus on a search for an NMDA antagonist drug that would be not cause neurotoxic side effects, at the dosages needed to exert a beneficial neuroprotective effect.

Because of the prominent role one of the inventors herein (Olney) played, first in elucidating the neurotoxic effects caused by excessive quantities of glutamate (Olney 1969), then in clearly locating and identifying the types of measurable neuronal damage that were being inflicted by NMDA antagonist drugs in the brains of lab animals (Olney et al 1989), Olney's laboratories were frequently requested to evaluate the neurotoxic risks of new candidate drugs that were becoming the heavily-investigated “lead compounds” in the drug companies' efforts to find a safe NMDA antagonist.

However, in every such investigation, Olney's tests disclosed that, if an NMDA antagonist drug was used in a quantity sufficient to actually reduce excitotoxic brain damage following a stroke or other brain insult, then it also caused neurotoxic vacuoles. Olney's research repeatedly revealed that even weak NMDA antagonists (such as dextromethorphan, the well-known cough suppressant that has been used for decades in cough syrups) would begin causing neurotoxic effects, if administered at dosages sufficient to reduce excitotoxic brain damage following a hypoxic crisis.

In the final analysis, the problem was not that the drugs were too potent to be used safely; instead, the problem was in certain inherent and unavoidable traits of the neuronal circuitry inside the brain. Glutamate molecules and NMDA receptors are so heavily and deeply involved, in so many crucially important circuits and regions inside the brain, that if any NMDA blocker drug is administered at a dosage which is potent enough to substantially slow down the excitotoxic processes triggered by excess glutamate accumulation inside a brain region suffering from a hypoxic crisis such as a stroke or cardiac arrest, then that protective dosage of that NMDA blocker drug will also be potent enough to trigger major disruptions and derangements in other crucially important neuronal circuits, as well.

In other words, because of the ubiquity of the NMDA receptor system inside the brain, any drug which blocks NMDA receptors at levels that can help suppress excitotoxic cell death, after a brain crisis, will also trigger major disruptions and derangements in other neuronal circuits. These disruptions and derangements were of such a type and magnitude that they inevitably led to toxic damage, affecting specific neurons in the cerebral cortex which perform important “clearinghouse” or “switchboard” functions.

In view of those consistent and repeated results from dozens of different drug tests on animals, Olney concluded that by far the best way, and probably the only realistic way, to reduce and prevent the neurotoxic side effects of NMDA antagonists was not by developing better NMDA antagonists, but by instead developing ways of coadministering NMDA antagonists along with other classes of drugs, which were eventually termed “safener” drugs, borrowing an old chemical term that had previously been used in the herbicide industry.

The first class of drugs that were discovered to block the toxic side effects of NMDA antagonist drugs were drugs that suppress activity at muscarinic receptors, which are a subclass of receptors activated by acetylcholine (ACh). These drugs, called “anti-cholinergic” or “anti-muscarinic” drugs, are discussed in the next section.

The Cholinergic System; Anti-Muscarinic Drugs

The “cholinergic” system, which uses acetylcholine (ACh) as its neurotransmitter, is another highly important excitatory system; the glutamate and cholinergic systems are the two most prevalent and important excitatory systems in any mammalian nervous system. Reviews of the physiology and activities of the cholinergic system include Aprison et al 1996, Zimmermann et al 1996, Everitt et al 1997, Buerkle et al 1997, Weinstock 1997, Kubo 1998, Israel et al 1998, and Oda 2000. Reviews of drugs used to modify activity at cholinergic receptors include Sullivan et al 1995, Iversen 1997, Geroldi et al 1997, Patocka 1998, and Tune et al 1998.

The two major classes of cholinergic receptors are called muscarinic, and nicotinic. Muscarinic receptors are relevant to this invention, and they have been further subdivided into classes designated as m1, m2, m3, m4 and m5 receptors. Drugs that suppress activity at one or more types of muscarinic receptors are well-known, and have played significant roles in medicine for decades.

Drugs that suppress activity at cholinergic receptors are often called anti-cholinergic drugs, which means the same thing as “cholinergic antagonists.” Similarly, drugs that suppress activity at muscarinic receptors are referred to herein as anti-muscarinic drugs. The term, “selective anti-muscarinic drug,” refers to an anti-cholinergic drug with substantially greater affinity for one or more types of muscarine receptors than for nicotine receptors.

A specific class of selective anti-muscarinic drugs have molecular structures that are referred to as aryl-cyclo-alkanol-amines (referred to by the acronym, “ACAA” drugs). As indicated by that name, and as shown by the chemical structures below, these drugs have an aryl (benzene or similar) ring, and a non-aromatic ring (such as cyclo-hexane), coupled to a short-chain alkyl group which has a pendant hydroxy group (i.e., an alkyl-alcohol, or alkanol). The alkanol component is also bonded to an amine structure (which is a heterocyclic ring in each of the compounds shown below, but which is not cyclic in various other known ACAA compounds).

| | |
|---|---|
| Procyclidine | Trihexyphenidyl |
| Biperiden | Triperiden |

The chemical structures of other anti-cholinergic ACAA's are listed in various editions of Goodman and Gilman, eds., The Pharmacological Basis of Therapeutics.

ACAA drugs such as procyclidine and biperiden were used for decades as anti-cholinergic agents, for two main purposes: (i) to treat patients suffering from Parkinson's disease; and, (ii) to treat patients who began suffering from Parkinson-like symptoms (such as muscular rigidity) as side effects of some of the earlier classes of anti-psychotic drugs that were used in the 1960's and 1970's (see AMA Drug Evaluations, 1971).

In recent years, these ACAA drugs have fallen into relative disuse, for two reasons. First, newer and better drugs have been developed for treating Parkinson's disease. And second, improved antipsychotic drugs have become available which do not cause the Parkinson-like side effects caused by earlier generations of antipsychotic drugs; therefore, anti-cholinergic drugs such as the ACAA drugs are no longer needed to control Parkinson-like side effects of other drugs.

Accordingly, ACAA drugs no longer receive the attention they received in earlier editions of Goodman and Gilman (the most widely used reference work of its kind). Most of the ACAA drugs named herein with shown, with their complete structures, in the fifth edition (1975) of Goodman and Gilman, but they are no longer shown in any recent editions. Examples of such older ACAA drugs that are no longer discussed in any detail in Goodman and Gilman include glycopyrrolate (ROBINUL), hexocylium (TRAL), oxyphenonium (ANTRENYL), tridihexethyl (PATHILON), and oxyphencyclomine (DARICON).

In addition to various drugs which fall squarely within the aryl-cyclo-alkanol-amine category, various other drugs with known anti-muscarinic activity have similar chemical structures, but do not fall squarely within the definition of “aryl-cyclo-alkanol-amines”. Examples include mepenzolate (sold under the trademark CANTIL), piperodolate (DACTIL), isopropamide (DARBID), thiphenamil (TROCINATE), adiphenine (TRASENTINE), dicyclomine (BENTYL), and poldine (NACTON). The structures of several such compounds are shown below, and others are shown in sources such as the Merck Index, older editions of Goodman and Gilman, etc. Drugs such as these are referred to herein as “ACAA analogs”. They are discussed in more detail below, under the subheading “ACAA Analogs and Derivatives.” All references below to ACAA drugs as a class are deemed to also refer to related analogs and derivatives of ACAA drugs, but only to analogs and derivatives which have certain essential properties which make them of interest herein for use in pain relief.

Several historical development also deserves attention in any analysis of ACAA compounds which may be useful as pain-relieving drugs. As briefly mentioned above, about 10 years ago, one of the Applicants herein (Olney) discovered that a number of well-known anti-cholinergic drugs (including but not limited to some ACAA drugs, such as procyclidine and biperiden) have a property that should have been of considerable interest to a number of companies and researchers that were actively trying to find ways to safely manipulate and modify activity at NMDA receptors. These anticholinergic drugs, if coadministered together with a potent NMDA antagonist that has severe neurotoxic side effects, will prevent the toxic side effects of the NMDA antagonist. That discovery (published in Olney et al 1991, and in U.S. Pat. No. 5,034,400, Olney 1991) was the first discovery and demonstration of a “safener” agent that could reduce and even eliminate the neurotoxic side effects of potent NMDA antagonists.

However, those 1991 reports were puzzling and apparently paradoxical, when viewed in light of an earlier published report. Several years earlier, in Olney et al 1987, the same research team had reported that several anti-cholinergic ACAA drugs also have significant NMDA antagonist activity.

The paradox was this: if an ACAA drug has NMDA antagonist activity, and if it is administered together with another drug that has even stronger NMDA antagonist activity, a logical person who understands the neurotoxic side effects of NMDA antagonist drugs would expect that the combined effect of administering two NMDA antagonist drugs, together, would be to increase (i.e., aggravate and worsen) the toxic side effects that NMDA antagonist drugs are known to cause. However, the research results clearly showed the opposite: all signs of the neurotoxic side effects of the potent NMDA blocking drug were abolished, when an ACAA drug having both anti-cholinergic and NMDA antagonist activity was administered together with the potent NMDA blocking drug.

When this latter finding was published (Olney et al 1991), other researchers working in this field considered it contradictory and did not know how to interpret it. It appeared that either Olney's initial claim, in 1987, that ACAA drugs have NMDA antagonist effects must be wrong, or his subsequent claim, in 1991, that ACAA drugs counteract NMDA antagonist effects, must be wrong. Thus, both sets of results were treated with skepticism, which is not uncommon in the world of science when a finding that appears to contradict conventional wisdom has been published by only one research team.

The attitude of skepticism referred to above is evidenced by the fact that, in the many years since the Olney et al 1987 report, there have been no publications from any other laboratories reporting any effort to confirm, deny, or further explore any of the implications of those findings. This is a curious and powerful commentary, when viewed in light of the two facts: (1) during the last two decades, drug companies have spent hundreds of millions of dollars, trying to find or develop drugs that can block NMDA receptors without causing neurotoxic side effects; and, (2) it is widely recognized and generally agreed that if non-toxic NMDA blockers could be created or discovered, they would have tremendous potential for treating a wide range of neurological conditions, including stroke, epilepsy, head trauma and neuropathic pain.

Nevertheless, despite the implication of the findings reported in Olney et al 1987, neither academic nor industry researchers attempted to confirm, deny, or even explore the possibility that ACAA drugs might be able to block NMDA receptors. To the best of the Inventors' knowledge and belief, no studies have ever been reported in which ACAA drugs were tested for efficacy in treating any neurological disorder in which excitotoxic brain damage might be involved (such as stroke, cardiac arrest, head trauma, etc.), or in which ACAA drugs were tested for efficacy in treating neuropathic pain, or any other chronic pain condition.

Skepticism regarding whether ACAA drugs are NMDA antagonists, and whether ACAA drugs can relieve neuropathic pain, has also been powerfully fueled and supported by two other factors.

The first factor is this: many laboratories have reported that NMDA antagonists are indeed effective in relieving neuropathic pain, in tests using lab animals (e.g., Davar et al 1991) and humans (e.g., Kristensen et al 1992). Based on those actual test results, and based on the growing understanding of the roles that NMDA receptors play in neuropathic pain, there is a generally shared assumption that, if a safe NMDA antagonist drug were available to the public, it likely would be very useful, indeed, in relieving at least some types of neuropathic pain, or other chronic pain. The problem is not based on any concern that NMDA antagonists would not be effective in relieving pain; the problem, instead, is that the ones that have been shown to work effectively have unacceptably toxic side effects.

That factor must be placed directly alongside the second factor: millions of people have been treated with ACAA drugs, over a span of decades. Many of those people (including many elderly patients) suffered from a wide range of ailments, some of which were painful. And yet, to the best of the Inventors' knowldege and belief, no evidence (not even anecdotal evidence) has ever been published, indicating that ACAA drugs could provide any relief from neuropathic pain, or any other kind of pain.

If ACAA drugs do indeed have a substantial level of NMDA antagonist activity, one would assume that their ability to reduce pain, based on that NMDA antagonist activity, would have been noticed by someone, at some time, during the decades that these drugs were in widespread use.

It is clear, therefore, that Olney's assertions pertaining to the NMDA antagonist properties of ACAA drugs have been ignored or disbelieved, by those schooled in the field of neurological research, and by companies and researchers that are actively pursuing efforts to develop NMDA antagonist drugs.

Finally, as described in the following paragraphs, another well-known line of prior art also teaches directly away from this invention, and prevented those skilled in the art from suspecting that ACAA drugs would be effective in relieving neuropathic pain.

Prior Art: Muscarinic Agonists as Pain Relievers

Another line of prior art directly contradicts and teaches away from the current invention. A substantial number of prior art articles, authored by numerous researchers over a span of 50 years, teach that muscarinic agonists have substantial pain-relieving activity. That is crucially important, because muscarinic agonists are drugs that stimulate, rather than suppress, neurotransmission via muscarinic cholinergic receptors. Clearly, that activity is the exact opposite of the activity caused by muscarinic antagonists (suppressors), such as the ACAA drugs involved in this invention.

The relevant reports are reviewed in Poyhia 2000. Briefly, Flodmark et al 1945 reported, more than 50 years ago, that a drug called prostigmine has pain-relieving activity. Prostigmine is a “cholinesterase inhibitor”, i.e., it slows down cholinesterase enzymes, which otherwise will rapidly break apart ACh molecules after they are released from ACh receptors. By preventing cholinesterase enzymes from quickly degrading ACh molecules, prostigmine increases the quantity of ACh that is present in the extra-cellular fluids which bathe cholinergic receptors.

This increase in ACh concentrations leads directly to an increase in ACh receptor activity. Accordingly, prostigmine is a cholinergic agonist; it has the opposite effect of a cholinergic antagonist, such as procyclidine. And yet, it was reported to be a pain-relieving drug.

That report was largely ignored for some years. Then, interest in the medical effects of tobacco and cigarettes (which contain nicotine, which triggers the nicotine class of cholinergic receptors) began provoking more research into the neurological and medical effects of cholinergic agonists. That led to a second round of reports which described the pain-relieving properties of cholinergic agonists (Mattila et al 1968; Bannon et al 1971).

Cholinesterase inhibitors, which increase the concentrations of ACh in extracellular fluids, are completely non-specific; they cannot selectively activate only certain subclasses of cholinergic receptors, while leaving other subclasses alone. Therefore, if tested in dosages that were high enough to provide substantial pain relief, they had two important shortcomings: (i) they caused intolerable levels of unwanted side effects, and (ii) their duration of pain relief was short, and wore off quickly. So, they did not merit or establish serious or widespread use as pain-controlling drugs, in the human clinic.

However, as more years passed, three other developments eventually led back to cholinergic agonists as pain-relieving drugs. The first development was the creation of receptor-specific selective agonists, which could target and activate only certain subtypes of cholinergic receptors, without activating or bothering other subtypes. As a general rule, the problem of unwanted side effects will usually decrease, and lower doses of a compound of interest can be used, if a more highly selective agent can be used, which does not manipulate and alter as many different types of neuronal receptors as a non-selective drug.

The second development was the expanding use of “intrathecal” injections (i.e., injection of drugs directly into the spinal fluid that is contained by the dural and arachnoid membranes that surround the spinal cord) and “epidural” injections (i.e., injection of drugs into the space adjacent to the dural membrane). To some extent, expanded use of spinal anesthesia was being driven and promoted by its increasing use in women who were delivering babies. It was widely recognized that one of the primary benefits of spinal injections, in women in the throes of labor, was that the drug did not enter the mother's circulating blood in a substantial quantity, and therefore posed no risk of affecting the vital functions of the baby that was being born.

The third development grew out of the expanding attention that was being given to pain management in terminally ill patients, especially cancer patients. As improvements in chemotherapy began keeping gravely ill cancer patients alive longer and longer, it became more important for pain management specialists to be able to treat such patients for prolonged periods.

As those factors converged, more anesthesiologists began administering more anesthetics via intrathecal or epidural routes. As they did, they realized that a number of old anesthetic drugs, which had caused too many undesired side effects or which had disappeared too quickly when administered using the old modes of administration, might deserve to be reevaluated using intrathecal administration, since a drug injected directly into the spinal region remained largely restricted to that region, and would not pose substantial risks of adverse symptoms.

Accordingly, anesthesiologists began using intrathecal and epidural injections to re-evaluate the same types of cholinesterase inhibitors that had caused too many side effects when administered systemically back in the 1960's. This led to articles such as Collins 1995, entitled, “Spinally administered neostigmine—something to celebrate,” in the journal Anesthesiology.

More than a dozen other articles, reporting similar results, were published during 1995-2000; as noted above, they are cited and summarized in Poyhia et al 2000, a short review article that is titled, “Cholinergic mechanisms of analgesia.”

The crucial point that must be recognized about all of those articles and all of the research they describe is this: they all report the successful and effective use of cholinergic agonists (i.e., cholinergic receptor stimulants), for treating pain.

By contrast, this invention teaches the exact opposite. This invention teaches that cholinergic antagonists (i.e., cholinergic receptor blockers) can be used safely and effectively for treating pain—but only if combined with a second drug that has a completely different activity, at a completely different set of neuronal receptors. Clearly, this current invention teaches in the exact opposite direction from the fairly extensive and accumulated prior art that is cited and summarized in Poyhia 2000.

The Adrenergic Transmitter/Neurohormone System

The other neurotransmitter-receptor system that must be taken into consideration, in the current invention, is called the “adrenergic” system. This system is discussed in Kandel et al, editors, Principles of Neuroscience, or in the Encyclopedia of Neuroscience (first edition, edited by Adelman, 1987, or second edition, edited by Adelman and Smith, 1999).

Very briefly, the two molecules that serve as transmitters in the adrenergic system are norepinephrine (also called noradrenalin) and epinephrine (also called adrenalin). The receptors that are triggered by these molecules are divided into two classes, designated as alpha (α) and beta (β). β-adrenergic receptors are not of any particular interest herein; they are reviewed in, e.g., Strosberg 1995 and Liggett 2000.

α-adrenergic receptors are subdivided into the α1 and α2 classes. The α2 subclass, which is of direct interest herein, has been further subdivided into A, B, C, and D classes, but “little is currently known about the specific function mediated by the various subtypes” (Bylund 1995). Reviews that focus on the α2 receptor system include Stornetta et al 1995, Saunders et al 1999, and Rosin 2000.

Reviews that focus on drugs which react with α1 and/or α2 receptor subtypes include Eisenach et al 1996, Kamibayashi et al 1997 and 2000, Newcorn et al 1998, Frishman et al 1999, and Cotecchia et al 2000.

Two facts about α2 adrenergic agonist drugs (referred to herein simply as α2 agonists, for convenience) should be recognized and emphasized, since both facts are directly relevant to this invention. One fact is this: α2 agonist drugs have sedating effects; indeed, they are often referred to as “sedative-hypnotic” drugs (e.g., Levanen et al 1996). As such, they are used in both veterinary and human surgery, to help boost the depth and quality of anesthesia provided by a drug such as ketamine (e.g., Verstegen et al 1989; Nevalainen et al 1989; Moens et al 1990; Levanen et al 1996; Handa et al 2000). The ability to relax a patient or pet, and take them deeper into anesthesia while also minimizing certain unpleasant side effects of ketamine anesthesia (such as psychotic “emergence reactions”) is useful and desirable, in anesthesia. However, it becomes a serious problem if a “sedating-hypnotic” drug must be taken every day to control chronic pain. The last thing a patient wants, when struggling with chronic pain, is to feel groggy, sedated, and semi-stuporous every day.

The second fact is this: α2 agonists substantially lower blood pressure. That is their main therapeutic use, i.e., reducing blood pressure in patients suffering from hypertension (high blood pressure). Reduced blood pressure can be useful in someone who needs it, but it can be very dangerous, and potentially even lethal, in someone who doesn't need it. Among other things, reduced blood pressure causes dizziness, which can be dangerous for anyone, and which is especially dangerous in elderly patients, since dizziness can lead to a fall, and a fall by an elderly patient often results in a broken hip or other severe injury.

Accordingly, both of those two facts teach directly away from the current invention, which involves the chronic use of α2 agonists to help control neuropathic pain.

Anyone who wishes to study the adrenergic system, or the α2 system in particular, can find a huge amount of information on it. During 1995 through 2000, more than 43,000 articles were published on the adrenergic system, including more than 3,700 review articles. This was more than twice as high as the number of articles written on the glutamate system during that same time span.

However, anyone approaching a study of the adenergic system for the first time should recognize and be forewarned that a huge amount of research is being done on that system, because (i) it is not yet well understood, and (ii) it is extraordinarily complex, multi-faceted, and apparently paradoxical in many respects.

A thorough analysis of all the reasons why the adrenergic system has been so difficult to figure out is beyond the scope of this text. However, a brief listing of some of the most relevant factors may help readers understand how and why the current invention appears to directly contravene and contradict several well-established lines of prior art. In highly condensed form, some of the more relevant complicating factors include the following:

1. Some parts of the adrenergic system function in a manner similar to conventional neurotransmitters, but other parts function more as hormones; accordingly, epinephrine and norepinephrine are sometimes called “neurohormones”. Those intermingled roles cannot be cleanly separated from each other.

2. The roles and effects of epinephrine and norepinephrine are very different, depending on which part of the body and nervous system is involved. Epinephrine (adrenaline) tends to have more powerful and direct effects, outside the CNS, but inside the CNS, norepinephrine has a much greater role. In addition, inside the CNS, adrenergic receptors play a predominantly inhibitory role; by contrast, outside the CNS, many (but not all) adrenergic receptors play excitatory roles.

3. At least some types of adrenergic receptors appear to be “inducible” and “regulated”. This means that the numbers and concentrations of adrenergic receptors on the surfaces of cells in various tissue types can change, substantially, in response to outside factors. For example, if abnormally high levels of adrenergic activation begin occurring in a certain part of the body, the affected cells will respond within a few hours, in ways that offset and counter-balance the surge of activity. These responses can include the deactivation and/or disappearance of existing receptors on cell surfaces, suppression of new receptor formation or expression, etc. These inducible responses make it even more difficult for researchers to obtain reliable and reproducible results from tests on animals or humans.

4. Finally, it must be recognized that the adrenergic system has major effects on numerous metabolic functions, but those effects can be manifested over widely varying time frames. Some effects are nearly instantaneous, such as a rapidly increased heartbeat rate, in response to the “adrenaline rush” that kicks in when an animal is confronted by danger. However, other effects can take hours, such as effects on gastrointestinal functions, urinary tract functions, etc., and still other effects can take weeks or months, such as muscle growth and fat accumulation. Therefore, testing of drugs that can affect one or more components of the adrenergic system tends to be unusually complex and risky, since adrenergic drugs tend to pose unusually high risks of triggering unwanted side effects.

For all of these reasons, research involving adrenergic receptor agonists, or antagonists, tends to be unusually difficult, and almost always requires low dosages, for the sake of safety, and to minimize unwanted side effects.

While norepinephrine (the natural neurotransmitter) activates both α1 and α2 adrenergic receptors, several selective α2 agonist drugs (including clonidine, iodoclonidine, guanabenz, guanfacine, xylazine, lofexidine, medetomidine, dexmedetomidine, tizanidine, rilmenidine, azepexole, alpha-methyldopa, and alpha-methylnoradrenaline) have been discovered which activate α2 receptors with substantially higher affinity than they have for α1 receptors.

As noted above, most α2 agonist drugs of commercial interest are used clinically as anti-hypertensive drugs, to help reduce blood pressure in patients suffering from hypertension. At least two α2 agonists, clonidine and lofexidine, also have been tested by researchers to evaluate their ability to help reduce withdrawal symptoms in people addicted to cigarettes or opiates (e.g., Washton et al 1983; Siegel et al 1985; Segal 1985; Green and Cordes 1989). α2 agonists have also been used as adjuncts in surgical anesthesia, as discussed above, and they have been injected into spinal regions, using intrathecal or epidural injections, for pain relief. However, they have never previously been used as daily treatments for chronic pain.

Indeed, the fact that α2 agonist drugs are commonly used by anesthesiologists, as sedative-hypnotic agents which can help relax patients before surgery, teaches directly away from the current invention. This invention requires chronic, daily use of a drug combination as disclosed herein. Therefore, known and substantial sedating and/or hypnotic activities of a drug being considered for this type of chronic, daily use would be highly adverse to the goal of identifying a non-sedating treatment for chronic pain.

Accordingly, one object of this invention is to disclose that ACAA drugs which have both (i) significant activity as a muscarinic antagonist, and (ii) significant activity as an NMDA antagonist, have some degree of efficacy in relieving neuropathic pain. However, any pain relief provided by these types of ACAA drugs, when used alone, is relatively brief, and is accompanied by unwanted side effects when administered at the dosages needed to substantially relieve neuropathic pain.

A much more important object of this invention is to disclose that when an ACAA drug which suppresses activity at both muscarinic receptors, and NMDA receptors, is co-administered with an α2 adrenergic agonist, these two agents mutually potentiate one another's ability to relieve neuropathic pain, in a manner which provides sustained and effective neuropathic pain relief, at a low dose of each agent that is below its threshold for producing any adverse side effects.

Yet another object of this invention is to disclose a unitary dosage form (such as a tablet, capsule, or skin patch) containing both an α2 adrenergic agonist drug and an ACAA drug, where each drug is present at a dosage which allows the combination to effectively control neuropathic or other chronic pain, without causing sedation, excessive reduction of blood pressure, cognitive or memory disruptions, or other undesired neurological or physiological side effects.

These and other objects of this invention will become more apparent through the following summary, drawings, and description of the preferred embodiments.

### SUMMARY OF THE INVENTION

This invention discloses that a combination of two drugs, from two different and previously unrelated categories, provides effective and long-lasting relief from neuropathic pain. Both drugs can be taken orally, in a convenient, painless, non-invasive manner that does not require injections.

One drug in this combination has an aryl-cyclo-alkanolamine (ACAA) chemical structure. At least some ACAA drugs have antagonist (receptor-blocking) activity at two entirely different classes of neuronal receptors: the muscarinic subclass of acetylcholine (ACh) receptors, and the NMDA subclass of glutamate receptors. ACAA drugs with this combination of receptor-blocking activities include procyclidine, biperiden, triperiden, and trihexyphenidyl. These drugs were used for decades as anti-Parkinson drugs because of their anti-cholinergic effects; however, they have largely been dropped and ignored, as better anti-Parkinson drugs have become available. These drugs were not previously suspected of having any pain-relief activity, but recent tests by the Applicants have shown that ACAA drugs can relieve neuropathic pain to some limited extent. However, at the doses required to relieve pain, they cause adverse side effects, and any pain relief is relatively brief and short-lived.

The other drug in the pain-relieving combination is an α2 adrenergic agonist, exemplified by clonidine. These drugs, normally used to control high blood pressure, have been used in a few cases for pain relief, but only by means of intrathecal injection (i.e., injection directly into spinal cord fluid, to avoid dangerous and potentially lethal reductions in blood pressure, which might occur if a high dose of an α2 agonist is injected into non-CNS tissue).

Tests described herein demonstrate that when an ACAA drug such as procyclidine is administered together with an α2 adrenergic agonist such as clonidine, these drugs mutually potentiate one another's neuropathic pain-relieving action, and provide potent and sustained neuropathic pain relief, even when each agent is administered at a low dosage that is below its threshold for causing adverse side effects.

Accordingly, these results indicate that combining selected drugs from these two different classes can provide safe and effective relief of neuropathic pain and possibly other types of chronic and/or intractable pain, at dosages which are so low that they do not pose serious risks of adverse side effects.

### BRIEF DESCRIPTION OF THE DRAWINGS

FIGS. 1 through 4 are graphs showing the results of tests using a standard model of neuropathic pain, involving the hind legs of rats, and their response to a warming light that shone on the sole of the animal's hindpaw. In all graphs, open circles indicate data from “ligated” limbs, in which the sciatic nerve was chronically irritated by a loop of suture material that was surgically placed and then tightened around the nerve. Filled circles indicate “control” data, from sham-operated limbs that did not suffer any chronic irritation. Rapid lifting of a ligated paw after the warming light commenced indicates that the paw was hyper-sensitized to pain signals.

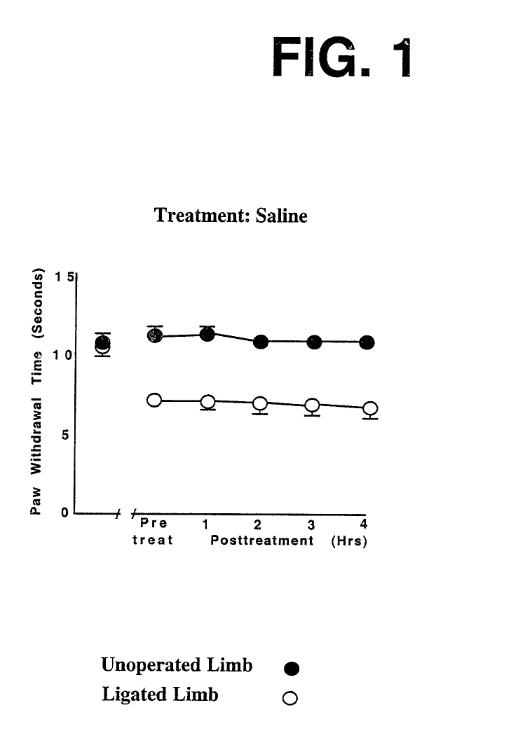

In graphs where the open circles are substantially lower than the filled circles, the drug treatment was not effective in controlling and reducing the neuropathic pain response. An example is provided in FIG. 1, which is a “baseline” control using a saline injection rather than a drug.

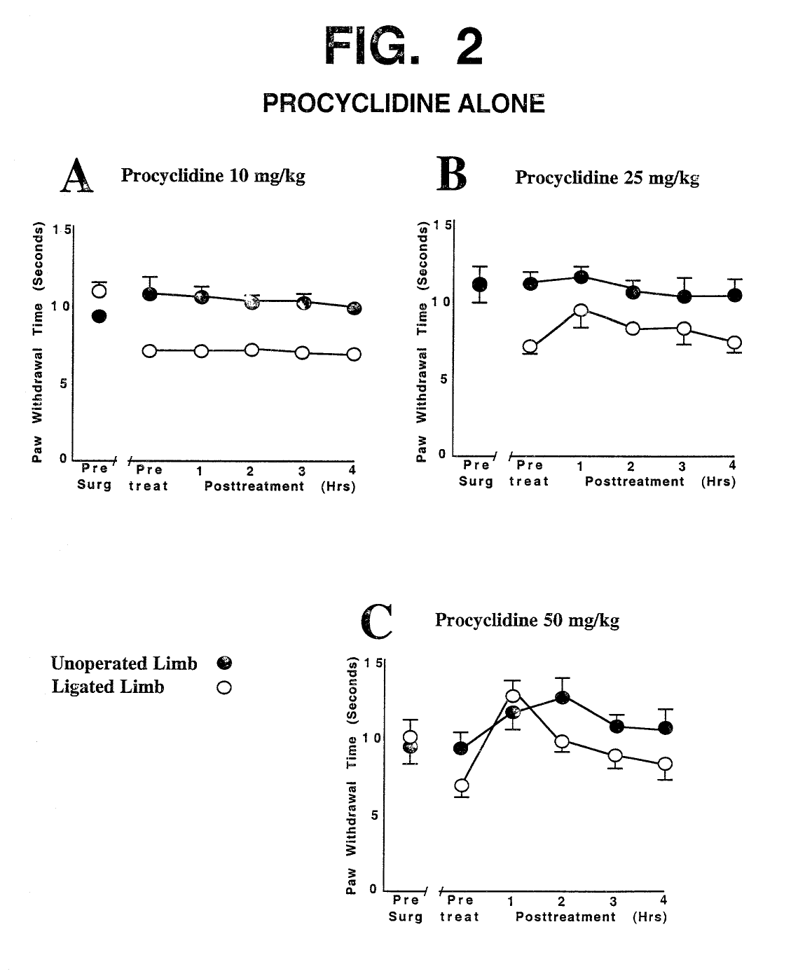

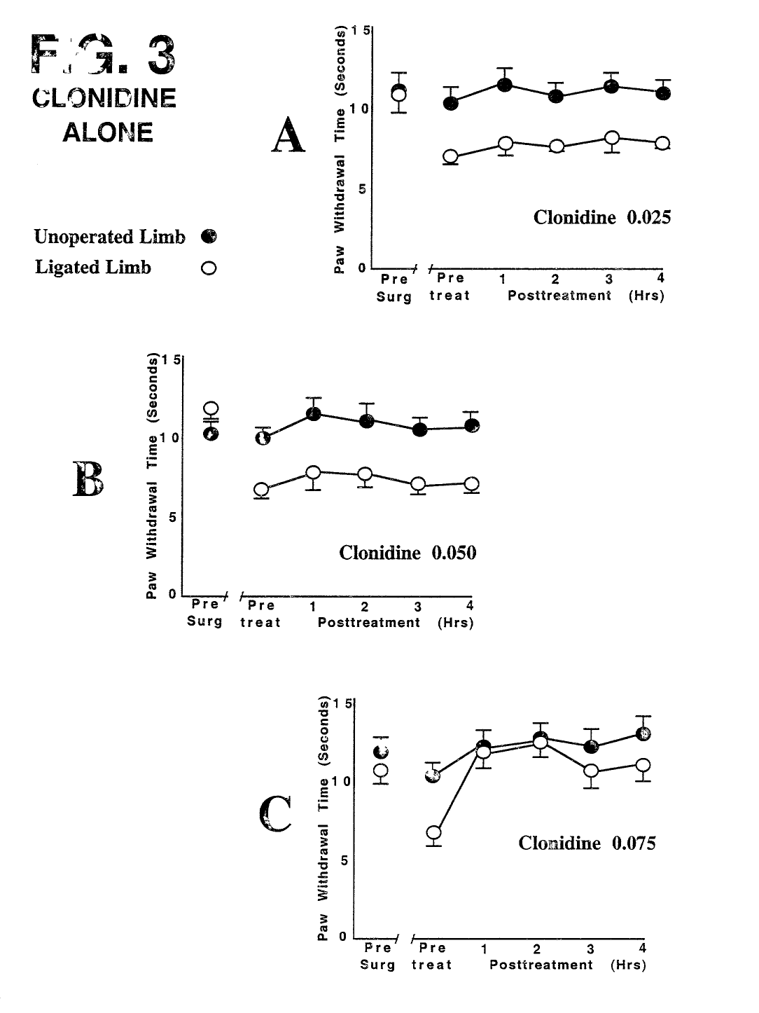

If a drug treatment was effective in reducing and controlling the neuropathic pain response, the vertical gap between an open circle (ligated limb) and a dark circle (non-ligated limb) was relatively small. The graphs in FIGS. 2 and 3 show that procyclidine and clonidine each provided some level of pain relief, but only at high dosages which caused behavioral side effects, and only for a short time.

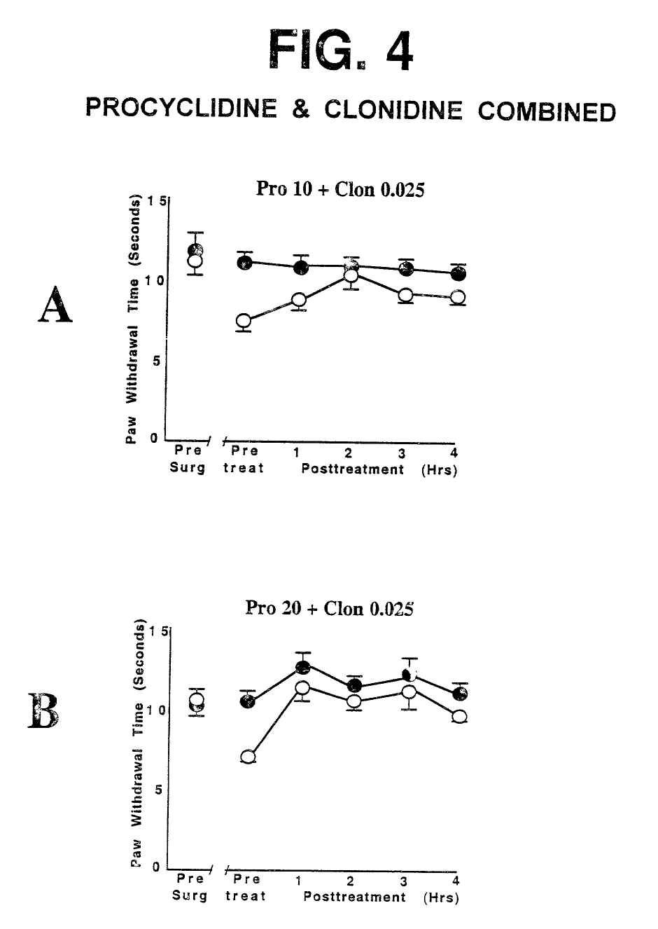

The graphs in FIG. 4 show that combinations of procyclidine and clonidine provided much better and longer-lasting pain relief than either drug could provide by itself. In addition, comparison of the effective dosages in these tests against the same dosages shown in FIGS. 2 and 3 show that the combination worked well, even at low dosages of both agents that were ineffective by themselves.

### DESCRIPTION OF THE PREFERRED EMBODIMENTS

This invention involves the use of a drug combination for treating chronic and/or intractable pain, such as neuropathic pain. The drug combination requires two distinct types of drugs, which are: (1) an aryl-cyclo-alkanolamine (ACAA) compound which has certain traits, described below; and, (2) an α2 adrenergic agonist drug. Examples of well-known and suitable ACAA drugs include procyclidine, biperiden, and trihexyphenydyl; examples of well-known and suitable α2 agonist drugs include clonidine, guanabenz, and lofexidine.

These drugs are likely to be used over long spans of time, such as months or even years, possibly for the entire remaining life of the patient. Instead of being a “cure” for neuropathic or other chronic pain, which would permanently alter and correct damaged or dysfunctional neuronal circuitry that causes the neuropathic or other chronic pain, this invention discloses a treatment and control regimen, which can help the affected patient suppress and control the chronic pain, with minimal unwanted side effects such as sedation.

Since these drugs are likely to be used on a daily basis (ranging from once per day, up to several times per day, depending on the severity of the condition being treated), both drugs should be in a form suited for noninvasive and painless adminstration. Such modes of administration include, for example, oral ingestion of a tablet, capsule, or liquid, or use of a skin patch or other “transmembrane” route.

Constituent 1: Aryl-cyclo-alkanolamines

To be suited for use as disclosed herein, an ACAA drug must have at least three distinct traits, and it preferably should have a fourth trait as well. A number of well-known ACAA drugs, including procyclidine, biperiden, triperiden, and trihexyphenidyl, have all four of these traits, and can be used as disclosed herein. Now that the utility of these drugs in treating neuropathic pain has been disclosed herein, any other known or hereafter-discovered ACAA drug (or ACAA analog or derivative, as discussed below) can be evaluated, using routine experimentation, to determine whether it has the traits described below.

First, an ACAA compound must be “pharmaceutically acceptable”. This term generally embraces the traits which make a drug suitable and practical for administration to humans, or to non-human animals if used in veterinary medicine. For example, such compounds must be sufficiently chemically stable to have an adequate shelf life under reasonable storage conditions, and they must be physiologically acceptable when introduced into the body by a suitable route of administration. The term “pharmaceutically acceptable” does not require a complete absence of any toxicity or unwanted side effects; instead, it implies that any toxic or other unwanted effects must be tolerable, and must be outweighed by some benefit which the drug can provide for at least some patients or animals.

Second, to be of interest herein, an ACAA compound must have a significant level of activity as an NMDA antagonist drug, i.e., a drug which can suppress activity at the NMDA subclass of glutamate receptors.

Third, for at least the current time and in the near future, an ACAA compound preferably should also have a significant level of activity as an anti-muscarinic drug, i.e., an anti-cholinergic drug with substantial affinity for muscarinic receptors. This trait, and the reason it is a strong preference at the current time, are discussed in more detail below.

And fourth, when co-administered with a suitable α2 adrenergic agonist drug such as clonidine, at suitable dosages which do not cause unacceptable side effects, it must be effective in providing sustained relief from neuropathic pain.

The reason why the third trait listed above (i.e., a significant level of anti-muscarinic activity) is preferred but not essential is as follows. Preferably, an ACAA drug which is being evaluated for use as disclosed herein should be “inherently” devoid of the neurotoxic side effects that are caused by potent NMDA antagonist drugs. This means that, if an ACAA drug with substantial NMDA antagonist activity is administered, by itself, to a test animal at high dosages, it will not cause vacuole formation, heat shock protein expression, or mitichondrial damage, which are several known manifestations of neurotoxic damage caused by NMDA antagonists such as MK-801, in neurons in certain parts of the brain that are highly vulnerable to such damage.

It is believed that this quality, of “inherent” safety, can be achieved by any ACAA drug that has substantial anti-cholinergic activity, since anti-cholinergic activity has been shown to block the toxic side effects of even the most potent NMDA antagonist drugs (e.g., Olney et al 1991, U.S. Pat. No. 5,034,400, Olney 1991).

This criterion (i.e., inherent absense of neurotoxic side effects) should be regarded as an important aspect of the drug combination, since inherent safety in any ACAA drug candidate will make it much easier to obtain the necessary approvals from the U.S. Food and Drug Administration (and similar agencies, in other countries) for public sale and use. This explains why the third criterion listed above (i.e., a significant level of anti-muscarinic activity) is strongly preferred at the current time, for any ACAA drug that is being evaluated for potential use as disclosed herein.

Nevertheless, anti-muscarinic activity, and the resulting trait of “inherent” safety is only preferred rather than essential, for an important reason. As described in U.S. Pat. No. 5,605,911 (Olney et al 1997), the α2 adrenergic activity exerted by the second drug in the pain-treating combination disclosed herein will, in most cases, provide an adequate level of safening activity for the ACAA comcpound, when the two drugs are coadministered together.

Questions of inherent safety, and the relative strengths of the muscarinic antagonist vs. NMDA antagonist activities of a candidate ACAA compound, may become important in the future, since analogs and derivatives of one or more known ACAA drugs (such as procyclidine, biperiden, etc.) can be developed which will have greater levels of NMDA antagonist activity, and lower levels of muscarinic antagonist activity. Because of the highly important role of NMDA receptors in neuropathic pain, it is likely that ACAA analogs and derivatives which have more potent and/or longer-lasting NMDA antagonist activity are likely to have better efficacy in reducing and controlling neuropathic pain than currently-known ACAA compounds (such as procyclidine, biperiden, etc.). In other words, analogs and derivatives of currently-known ACAA compounds which have stronger NMDA blocking activity, will offer good candidates for evaluation as disclosed herein, in combination with an α2 adrenergic agonist, regardless of whether such ACAA analogs or derivatives have the same, greater, or lesser activity at muscarinic receptors.

Indeed, it may well be possible, in the future, to develop and commercialize ACAA compounds or analogs which have little or no significant anti-muscarinic activity. As of March 2001, not a single already-known NMDA antagonist has ever been approved for use in humans by the U.S. Food and Drug Administration, despite the potentially large and important medical benefits they may be able to provide. However, as more bi-functional drugs are developed with the full knowledge that they do indeed have substantial levels of NMDA antagonist activity, but they are “inherently safened” due to a second type of neuronal receptor activity, it is possible that certain regulatory and other institutional biases will evolve and adapt accordingly, and greater interest will be shown in drug combinations having a first drug which is openly recognized to have toxic side effects, if those toxic side effects can be adequately prevented by a second “safener” drug in a two-drug combination.

If and when that type of attitude emerges as a driving force (rather than a major impediment) in new drug development, then ACAA drugs or analogs with little or no anti-muscarinic activity, but with substantial NMDA antagonist activity, may become commercially viable. Indeed, such drugs may even be preferred, since they are likely to pose lower risks of unintended side effects. However, that day has not arrived, and does not appear imminent. Accordingly, for the time being, a substantial level of anti-muscarinic activity is regarded as a strongly preferred criterion for an ACAA compound to be of interest herein, since anti-muscarinic activity will make the compound “inherently safe,” and will prevent the neurotoxic side effects that would otherwise be caused by NMDA antagonist activity. This will allow its safety and lack of toxic side effects to be clearly shown even when the drug is administered by itself, without an α2 agonist drug to accompany it.

Constituent 2: Adrenergic Agonists

In addition to clonidine (a prototypical α2 agonist, discussed above and used to generate the test results shown in the Figures), various other known drugs have been developed which have selective affinity for α2 adrenergic receptors. Such drugs include iodoclonidine, guanabenz, guanfacine, xylazine, lofexidine, medetomidine, dexmedetomidine, tizanidine, rilmenidine, azepexole, α-methyldopa, and α-methyl-noradrenaline. Any of these drugs, or any other α2 adrenergic agonist which is currently known or discovered in the future, can be tested for efficacy in reducing and controlling neuropathic pain or any other form of chronic and/or intractable pain, when coadministered with an ACAA compound having NMDA antagonist activity.

In general, “selective” or “highly selective” α2 agonists are preferred, since they pose lower dangers of unwanted side effects. For example, clonidine is regarded as a selective α2 agonist, since its affinity for α2 adrenergic receptors is significantly higher than its affinity for α1 adrenergic receptors. However, clonidine is also known to have significant affinity for α1 receptors.

Accordingly, clonidine (which is one of the most well-known and widely used α2 agonist drugs) is regarded herein as a “benchmark” drug. It is a “selective” α2 agonist drug, but it is not regarded as a “highly selective” α2 agonist drug. The phrase “highly selective α2 agonist” refers to an α2 adrenergic agonist drug which has substantially higher selectivity for α2 receptors (and substantially lower affinity for α1 receptors) than clonidine. Published reports have stated that such “highly selective” α2 agonists include dexmedetomidine, guanfacine, and azepexole, and may also include lofexidine and various other α2 agonists as well.

In addition, it must be recognized that a major physiological effect of clonidine (and most other known α2 agonist drugs) is their ability to reduce blood pressure. That is their normal medical use, and it is quite useful in patients suffering from hypertension (high blood pressure). However, reductions in blood pressure are likely to be unwanted, and even potentially dangerous, in patients who do not suffer from hypertension, and who instead are suffering from neuropathic or other chronic pain. Accordingly, the phrase “α2 agonist which has little hypotensive side effect” refers to an α2 agonist which has less activity than clonidine, in reducing blood pressure. Published reports indicate that lofexidine and guanfacine both fall within this criterion, and various other α2 agonists may also qualify.

Although the Applicants are not aware of any publication that systematically ranks or quantifies numerous α2 agonists in terms of α2 selectivity, potency in reducing blood pressure, severity or absence of side effects, and ability to penetrate mammalian blood-brain barriers, such information is provided, in scattered form, in various locations (e.g., Goodman and Gilman 1990 at page 308; Ruffolo et al 1993 at page 264; Doze et al 1989 at page 75). In addition, evaluation of these relevant properties in any known or hereafter-discovered α2 agonist can be carried out with routine experimentation, using conventional procedures. Such tests include competitive binding assays to evaluate selectivity for α1 and α2 receptors; tests on both normal and hypertensive lab animals, to evaluate hypotensive activity; and analysis of CNS tissue following administration of radiolabelled drugs, to evaluate BBB permeability. Accordingly, based on the teachings herein, methods are provided for screening the α2 agonist drugs listed herein, or any other α2 agonist drugs which are currently known or hereafter discovered, and determining which ones will have the best profile of benefits vs. unwanted side effects, when coadministered with an ACAA drug which has NMDA antagonist activity, to relieve neuropathic or other chronic pain as disclosed herein.

It is not asserted that the drug combinations disclosed herein will be completely free of any and all side effects, in all patients. Instead, this disclosure asserts that: (i) this drug combination allows the use of relatively low dosages of both constituent drugs, when they are coadministered with each other; and, (ii) the use of relatively low dosages of both constituent drugs strongly indicates that any adverse side effects will be minimized, and will be fully acceptable to large numbers of patients. Indeed, one of the most remarkable and valuable traits of the drug combination disclosed herein is that these two classes of drugs, when administered together, appear to be highly potent and effective in reducing neuropathic pain, even when each drug is administered at dosages which are only a fraction of the dosages that begin to exert detectable behavioral or other effects in treated animals.

As used herein, the phrase “unacceptable side effects” refers to side effects which, in a particular patient or class of patients, rise to a level of unpleasantness which that patient (or class of patients) regards as outweighing the benefits of the treatment. Clearly, dosages and frequency of administration will vary substantially between different patients; patients who are suffering from nearly unbearable intractable pain are likely to take high doses of both drugs, four or more times each day; by contrast, patients who are suffering from minor annoyances will take lower dosages, only once or twice each day. Accordingly, the acceptability of any side effects a drug combination as disclosed herein might cause, in a particular patient or class of patients, must be viewed in light of the pain-relieving benefits it provides for that patient or class of patients.

Animal Test Design and Results

All of the graphs in FIGS. 1-4 pertain to the effects of various drug treatments on measurable pain-sensitive responses in test animals (rats). FIG. 1 shows the results of saline controls, which provided no pain relief. FIGS. 2 and 3 displays the results of a single drug (procyclidine or clonidine). FIG. 4 displays the results of the procyclidine-plus clonidine combination, in two different dosages.

In all experiments, one hind limb of the rat has undergone a surgical operation in which a loop (“ligature”) of suture material was placed around the sciatic nerve and gently tightened. Over the course of several days, this caused chronic irritation to the nerve, driving it into a “hyper-sensitized” condition of the type which occurs in damaged neuronal circuits that cause neuropathic pain in humans. The other hind limb of each rat did not receive any operation, and served as a control condition.

After the nerve ligation operation, a waiting period of 8 days elapsed, to allow the ligated limb to reach a peak of hypersensitized chronic irritation of the ligated sciatic nerve. This sciatic nerve model was first described by Bennet and Xie 1988, and is widely regarded as a useful animal model for studying neuropathic pain.

To test the degree of sensitivity in the ligated limb vs. the unoperated limb, a beam of light (thermal stimulus) is applied to the soles (i.e., the undersurface area which is not covered by fur) of the hind paws, by shining a focused beam of light onto the sole, through the glass floor of a testing chamber. This beam was sufficiently hot to generate some discomfort, without posing any risk of burning the paw. The number of seconds that elapsed after commencement of the thermal stimulus, until the rat lifted up the paw, were detected electronically and recorded automatically.

Each limb is tested initially for thermal pain sensitivity, before the ligation operation is performed (pre-surgery), to ensure that both limbs had the same degree of pain sensitivity. After 8 days following surgery, each limb was tested again, before any treatment was given (pre-treatment), to ensure that the nerve ligature successfully provoked a hyper-irritable condition in the ligated limb. After drug treatment (at time=0, on the graphs), each limb was tested for thermal pain sensitivity at hourly intervals, for 4 hours.

The graph in FIG. 1 depicts the level of pain sensitivity in each limb under control conditions, when a saline injection instead of an active drug was administered. In all graphs, open circles indicate data from ligated limbs, and filled circles indicate control data, from sham-operated limbs that did not have any ligatures to cause chronic irritation.

Rapid lifting of a ligated paw indicates that the paw was hypersensitive. If a large vertical gap occurs between the ligated limb (open circle) and the control limb (closed circle), such as shown in FIG. 1 at all times, this indicates that (i) the ligated paw was indeed suffering from a hyper-irritable neuropathic pain condition, and (ii) the drug treatment was not effective in reducing and controlling the response to the painful stimulus. After saline (control) treatment, there was no change in pain sensitivity in either limb, as indicated by the fact that the open circles (ligated limbs) and dark circles (unoperated limbs) continued to be widely separated, consistently showing more rapid paw withdrawals in the ligated limbs, following thermal stimulus, at each hourly interval.

The graphs in FIG. 2 show that intraperitoneal injection of procyclidine, at three different dosages, did not provide adequate and lasting relief from pain sensitivity. At the lowest dosage used (10 mg/kg, shown in FIG. 2A), the procyclidine had no apparent effect at all. At 25 mg/kg, shown in FIG. 2B, the procyclidine had some effect after 1 hour, but that response in ligated limbs did not reach or even closely approach the response times in unoperated limbs; in addition, even that mild effect shown at 1 hour dropped off substantially within 2 hours.

At the highest dosage tested (50 mg/kg, shown in FIG. 2C), complete relief from neuropathic pain was seen at the 1 hour testing time. However, this level of pain relief dropped off substantially by the second hour; and, just as importantly, a comparable dose in humans would very likely cause substantial discomfort (such as dry mouth, blurred vision, and gastrointestinal disturbances) in at least some patients, due to the strong anticholinergic action of procyclidine.

The graphs in FIGS. 3A, 3B and 3C show the pain sensitivity in each limb, following intraperitoneal injection of clonidine at 0.025 mg/kg, 0.05 mg/kg or 0.075 mg/kg, respectively. At the two lower doses, clonidine showed no significant relief of pain. At the highest dose, there was significant and relatively sustained neuropathic pain relief; however, at that dosage of clonidine, the rats displayed significant sedation and loss of motor activity and control, as described in Example 5. In at least some and probably even most humans, a comparable dose of clonidine would very likely produce serious unwanted sedation, and a lowering of blood pressure to an unacceptable and potentially dangerous degree.

The graphs in FIGS. 4A and 4B show that when procyclidine and clonidine were administered together, the combination provided sustained relief of neuropathic pain. Just as importantly, these graphs show that effective and sustained pain relief was provided at relatively low dosages, which did not cause any significant reduction in pain when each drug was administered by itself. In FIG. 4A, a very low dose of procyclidine (10 mg/kg) plus a very low dose of clonidine (0.025 mg/kg) caused a substantial decrease in the vertical gaps between the two curves. While peak relief was seen at 2 hours, a substantial degree of pain relief was still evident at 4 hours, when the testing stopped. FIG. 4B shows that if the dose of procyclidine was increased to 25 mg/kg (which was still ineffective, by itself) and the dose of clonidine was held to a low dose (0.025 mg/kg), effective and sustained relief of neuropathic pain was achieved.

Modes of Administration

The compositions of this invention comprise mixtures of an alpha-2 (α2) adrenergic agonist, and an aryl-cyclo-alkanolamine (ACAA) drug which has antagonist activity at NMDA receptors, and preferably at muscarinic receptors as well. The two components act together in a synergistic and potentiating manner, causing the mixture to provide better and longer lasting non-sedating pain relief than either drug can provide without the other drug. This allows each component to be administered at a relatively low dosage, and avoids or minimizes any undesired side effects.

The compositions of this invention may be administered by any suitable route which will introduce the intended drug(s) into the bloodstream. As noted above, preferred modes should use painless non-invasive means, such as oral ingestion of tablets, capsules, or liquids, or transmembrane routes, such as skin patches, nasal sprays, lozenges, penetrating ointments, etc. Intramuscular, intravenous, or other forms of injection, as well as subcutaneous implantation of slow-release devices or formulations or osmotic mini-pumps, are also possible, but they are less convenient and more painful and troublesome than noninvasive modes such as pills or skin patches.

All such modes of administration are all well known in the pharmaceutical arts, and typically require a pharmaceutically acceptable carrier in addition to the active ingredients. In making the pharmaceutical mixtures disclosed and claimed herein, the active ingredients will usually be mixed with and diluted by a carrier, or enclosed within a carrier such as a capsule. When the carrier serves as a diluent, it may be a solid, semisolid or liquid material which acts as a vehicle, excipient or medium for the active ingredients. Thus the composition can be in the form of tablets, pills, powders, lozenges, chewing gum, cachets, elixirs, emulsions, solutions, syrups, suspensions, aerosols (as a solid or in a liquid medium), ointments containing for example up to ten percent by weight of the active compound, soft and hard gelatin capsules, suppositories, sterile injectable solutions, and sterile packaged powders.

For oral administration, the compositions of this invention can be admixed with carriers and diluents molded or pressed into tablets, or enclosed in gelatin capsules, or otherwise loaded into digestible plastic or other capsules. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propyl-hydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient, by employing procedures well known in the art.

The compositions are preferably formulated or packaged in a unit dosage form, each dosage unit containing an effective amount of both (i) one or more alpha-2 adrenergic receptor agonists, and (ii) a selected ACAA compound. The term “unitary dosage form”, as used herein, has two meanings. One meaning refers to physically discrete units (such as capsules, tablets, or skin patches) which are suitable as unitary dosages for human subjects (or for pets, etc.) wherein each unit (each pill, skin patch, etc.) contains a predetermined quantity of each of the two active drugs discussed herein. In this type of unitary dosage form, the dosages of both drugs are predetermined, and are designed to safely produce the desired therapeutic effects, in most patients.

The second meaning of “unitary dosage form” refers to a liquid, dry powder, or similar formulation which is not divided into discrete units, but which contains each of the two active drugs discussed herein at predetermined concentrations which allow desired dosages of both drugs to be dispensed conveniently by referring to a fixed volume of the liquid or powder. As one example, a liquid containing two active drugs in a carrier can be provided in a unitary dosage form, if the desired dosage of each drug can be taken by ingesting a fixed quantity (such as a single teaspoon, tablespoon, etc.) of the liquid. Similarly, a powdered formulation of two drugs can be provided in a unitary dosage form, if the desired dosage of each drug can be taken by stirring a teaspoon, tablespoon, or “scoop” (if a plastic scoop is provided in the container) of the material into a suitable liquid which is then swallowed.

Alternately or additionally, non-divided preparations such as liquids, aerosols, or powders can be packaged inside unitary-dosage container devices which are designed to dispense a predetermined volume of material each time the device is used. Examples of unitary-dosage dispensing containers include: (i) inhaler devices, which release a predetermined amount of medicine (such as for treating asthma) into the mouth or nasal sinuses each time the device is squeezed; and, (ii) plastic bottles which are provided with a small chamber near the outlet, wherein the chamber is provided with a liquid-level marker that indicates how much liquid should be squeezed into the chamber for a proper dose of the liquid. By controlling the concentration of each drug in the liquid, aerosol, powder, or other preparation in a dispenser, unitary dosage of drug mixtures can be provided by such devices.

In either case, a “unitary dosage form” of a non-divided material such as a liquid, aerosol, or powder can be provided by controlling the concentrations of both drugs carried by the material, so that administration of a controlled volume of the material will provide the desired unitary dosage.

The amount of either drug, in a unitary dosage formulation, will depend on factors such as the severity of the pain condition being treated in a particular patient, and the the amount and potency of the other drug in the same formulation. These drug combinations are (or are likely to be) available only with a prescription from a physician; they are not available over-the-counter. Accordingly, the preferred dosage and mode of administration of the two drugs, in combination, will be under the control of a qualified physician, who can evaluate all relevant factors for any specific patient (including the age, weight, and response of the individual patient, the severity of the patient's symptoms, the chosen route of administration, etc.). In addition, it is standard practice in treatments of this nature for a physician to administer an initial dosage for a test period such as two weeks, and subsequently adjust the dosage depending on the results observed during the test period. Therefore, the dosage ranges provided below are intended to provide general guidance and support for the teachings herein, but are not intended to limit the scope of the invention.

In general, the preferred dosage of an α2 agonist will usually lie within the range of from about 0.001 to about 1000 mg, more usually from about 0.01 to about 500 mg per day. The amount of the ACAA drug will typically be within the range of from about 0.001 to about 1000 mg, more usually from about 0.01 to about 500 mg per day. Alternately, expressed in terms of mg/kg (i.e., milligrams of drug per kilogram of patient body weight), the dosage of α2 agonist normally will fall within a range of about 0.00002 to about 50 mg/kg of body weight, and most commonly will fall within a range of about 0.0002 to about 20 mg/kg body weight, while the dosage of the ACAA will normally fall within a range of from about 0.00002 to about 50 mg/kg of body weight, and most commonly within a range of about 0.0002 to about 20 mg/kg body weight.

The terms, “drug”, “agonist”, and “antagonist,” as used herein, includes so-called “pro-drugs” which are administered in a form that is known and intended to be metabolized, inside a patient's body, into a different form which has a specific desired activity. As an example, α-methyldopa is a pro-drug form of an α2 agonist; the dopa form is actively transported into the CNS, which is desirable, and then enzymes inside the CNS convert the dopa form into α-methyl-norepinephrine, which is the active α2 agonist form of the drug. In such cases, both α-methyldopa and α-methyl-norepinephrine would be regarded as α2 agonist drugs for the purposes of this invention.

Various salts and isomers (including stereoisomers) of the drugs listed herein can be used. The term “salts” can include alkali metal salts as well as addition salts of free acids or free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. Alkali metal salts or alkaline earth metal salts include, for example, sodium, potassium, calcium, or magnesium salts. All of these salts (or other similar salts) may be prepared by conventional means. The nature of the salt or isomer is not critical, provided that it is non-toxic and does not substantially interfere with the desired pharmacological activity.

ACAA Analogs and Derivatives

As noted above, a number of anti-cholinergic drugs are known which have structures that closely resemble ACAA drugs, but which do not fall squarely within the term “aryl-cyclo-alkanol-amines”. Examples include mepenzolate (sold under the trademark CANTIL), piperodolate (DACTIL), isopropamide (DARBID), thiphenamil (TROCINATE), adiphenine (TRASENTINE), and dicyclomine (BENTYL). Their structures are illustrated below, to show their similarity to ACAA drugs:

| | |
|---|---|
| Mepenzolate | Piperodolate |
| Osopropamide | Thiphenamil |
| Adiphenine | Dicyclomine |

These and similar drugs are referred to herein as “ACAA analogs”. The term “analog” is used herein in the conventional pharmaceutical sense, to refer to a molecule that structurally resembles a referent ACAA molecule (such as procyclidine, biperiden, triperiden, or trihexyphenydyl, but which has been modified in a targeted and controlled manner to replace one or more specific substituents of the referent molecule with an alternate substituent, thereby generating a molecule which is structurally similar to the referent molecule. Synthesis and screening of analogs, to identify slightly modified versions of a known compound which may have slightly improved traits (such as higher potency and/or selectivity at a specific targeted receptor type, greater ability to penetrate mammalian blood-brain barriers, etc.) is an approach that is well known in pharmaceutical chemistry.

Similarly, the term “ACAA derivative” is used herein to describe a compound which is derived from an ACAA compound, i.e., an ACAA compound is used as a starting reagent or intermediate, in the process used to create a similar but slightly different drug which resembles an ACAA compound but which does not fall squarely within the aryl-cyclo-alkanol-amine boundaries.

ACAA analogs and derivatives are covered by the claims below, but only if and to the extent that they have the four essential traits and activities of ACAA drugs which are of interest herein (i.e., pharmaceutically acceptable; substantial anti-muscarinic activity; substantial NMDA antagonist activity; and, therapeutic efficacy against neuropathic pain when co-administered with an α2 adrenergic agonist drug). If some particular ACAA analog or ACAA derivative does not have all four of those traits, it is of no interest herein, and it is not intended to be covered by the claims.

Issues of BBB Permeability

It is well-known that large numbers of NMDA receptors and α2 adrenergic receptors are present on neuronal surfaces both inside the CNS, and outside the CNS. A major difference and distinction between those two classes of neurons relates to the so-called “blood-brain barrier” (BBB), which exists in all vertebrate animals. Except for a few minor specialized classes of neurons (such as those referred to as “circumventricular organ” neurons), a general distinction between CNS versus non-CNS neurons is that most CNS neurons have their cell bodies (i.e., the thickest part of the neuron, which contains the nucleus) located in tissue that is protected by the BBB, while most non-CNS neurons (which are involved in neuronal circuits usually called peripheral, autonomic, sympathetic, parasympathetic, etc.) have their cell bodies located in tissue that is not protected by the BBB. However, since many types of both CNS and non-CNS neurons have axons and other fibers that pass through the BBB, to help implement CNS control over muscles, organs, and other tissues, it is not entirely accurate to refer to CNS neurons as being protected by the BBB while CNS neurons are not.

The BBB is not a single barrier in the form of a membrane surrounding the brain and spinal cord. Instead, it arises from the fact that the capillaries which service CNS tissue have special structures, with “tight junctions” between the endothelial cells that make up those capillary walls. These “tight junctions” prevent a wide variety of molecules from passing through the capillary walls in CNS tissue, and thereby prevent CNS neurons from being contacted and potentially disrupted by such molecules. Recent monographs and review articles that discuss the physiology and functioning of the BBB include Brightman et al 1992, Rubin 1999, Pardridge 1999, Kniesel et al 2000.

Since NMDA receptors and α2 adrenergic receptors are present in large numbers both inside and outside the BBB, it is not yet known whether or to what extent the benefits of the invention disclosed herein (such as longer duration of pain relief, etc.) might be improved (such as by achieving longer duration of pain relief, etc.), in one or more classes of patients, by using: (i) an ACAA drug or analog thereof which has somewhat lower, or somewhat higher, BBB permeability levels than a known ACAA drug such as procyclidine; and/or, (i) an α2 adrenergic agonist which has somewhat lower, or somewhat higher, BBB permeability levels than a known α2 agonist such as clonidine.

That additional knowledge, even if it may be able to someday enhance and improve some embodiments of this invention for some patients, is not essential to carrying out this current invention. Instead, this invention is already fully enabled, by the disclosure that certain combinations of already-known drugs having already-known properties can act synergistically, to provide effective, sustained, and apparently non-sedating relief from at least some types of neuropathic pain, even at low dosages of each drug.

Nevertheless, the possibility of further improving this invention should not be neglected, even at this very early stage of disclosure. Accordingly, one approach to improving this invention, possibly for large numbers of affected patients or possibly for only limited categories of patients, involves further analysis to evaluate the pain-relieving effects provided by specific drugs, combined as disclosed herein, that have greater or lesser BBB permeability than procyclidine and clonidine.

In this type of work, it should be recognized that various methods and models have been developed, for animal testing of BBB permeability; these are reviewed in, for example, Bonate 1995. In addition, early efforts to create in vitro (cell culture) models for evaluating BBB permeability of candidate drugs are discussed in Townsend et al 1995 and DeBoer et al 1999.

Other articles focus more specifically on various methods of getting candidate drugs through the BBB and into brain tissue. Most such methods generally fall into one of three categories:

(1) manipulating the BBB to render it more permeable for a brief period of time, such as by intravenous injection of an osmotic-manipulating solution shortly before an anti-cancer drug is injected into a patient with brain cancer;

(2) binding “passenger” compounds to antibodies that can bind to and exploit a BBB transport system such as the transferrin system; and,

(3) modifying candidate drug compounds, to increase their lipophilicity or otherwise increase their ability to penetrate the BBB.

All of these methods are discussed in various review articles, including Langer 1989, Abbott 1996, Begley 1996, Pardridge 1998, Kroll et al 1998, and Rochat et al 1999, and in numerous other research reports which are cited in these review articles. Clearly, for long-term use of a drug designed to control chronic pain, the third approach listed above holds the most promise.

Since it is a well-known principle that drugs which are relatively lipophilic tend to cross mammalian blood-brain barriers more readily and in higher concentrations, one of the most commonly used approaches to increasing BBB permeability of a candidate drug involves modifying a known compound (which can be regarded as the initial, starting, or “referent” compound; procyclidine and clonidine offer examples) in a manner that increases its BBB permeability, by modifying the known/starting compound in a manner that slightly increases its lipophilicity. This can be done by various methods known to those skilled in organic chemistry, such as by modifying pendant groups that are bonded to the central structure of a molecule. As examples, a lower alkyl group might be added as a pendant group to a referent compound; a slightly longer alkyl group might be substituted for one that is already present; or an atom or group with a lower degree of polarity might be substituted for a highly polar atom or group. Any of these substitutions (or various others, as discussed in articles such as Kroll et al 1998, Rochat et al 1999, etc., cited above) can create analogs that are more lipophilic, and that are likely to be more readily able to penetrate blood-brain barriers, than the starting/referent compound.

Conversely, if it is desired to create and evaluate candidate drugs that are less lipophilic, that can be done by a chemical modification which is the opposite of an approach listed above.

The lipophilicity of candidate compounds often can be predicted with fairly good accuracy by a skilled chemist, just by considering the constituents of the molecule, especially if the molecule is a related analog of another compound with a known level of lipophilicity. Regardless of the accuracy of such a prediction, the lipophilicity of any candidate drug can be measured using simple tests, such as measuring how much of the compound goes into the organic phase, and how much goes into the water phase, when a fixed amount of the compound is stirred into a container that contains both water and a non-polar organic solvent.

Accordingly, analogs and derivatives of any known or hereafter-discovered drug can be created which are either more lipophilic, or less lipophilic, than existing known drugs. Such analogs and derivatives offer good candidates for evaluation in animal tests, to identify candidate compounds that penetrate the BBB either in somewhat higher concentrations, or in somewhat lower concentrations, using methods such as described in Bonate 1995.

Numerous pharmaceutical companies have already patented many hundreds and possibly even thousands of analogs of their best-known ACAA drugs, and their best-known α2 adrenergic agonists. For various reasons, those compounds were not chosen for commercialization to treat Parkinson's disease (in the case of ACAA drugs) or hypertension (in the case of α2 adrenergic agonists). However, now that an entirely separate and different therapeutic use (or potential class of uses) for such drugs has been disclosed, the numerous already-known but not-previously-commercialized variants of the previously-commercialized ACAA drugs and α2 adrenergic agonists can be reevaluated, using no more than routine experimentation, to determine whether they may be more effective (longer lasting, fewer side effects, etc.) than the previously-commercialized ACAA drugs and α2 adrenergic agonists, in treating any particular type or class of neuropathic pain or other chronic or intractable pain.

### EXAMPLES

### Example 1

Test Procedures and Control Results

The first set of in vivo tests involved sciatic nerve ligation in rats, using procedures described in Bennett and Xie, Pain 33: 87-107 (1988). This test involves surgically placing and tightening a loop of suture material around the sciatic nerve in one hind leg of a rat; the other hind leg serves as a control. Within about a week, the ligated sciatic nerve becomes irritated and reaches a hyper-responsive “kindled” state, where it will respond rapidly to even a mild stimulus that is not painful to an untampered leg. As such, it offers a model of what happens in pain pathways that have become pathologically hypersensitized in a human suffering from neuropathic pain.

In these tests, on the 8th day after surgery, the rat is placed in a testing device which electronically measures how quickly or slowly it acts, in lifting up a paw in response to a standardized mild warming (thermal) stimulus. The warming stimulus is generated by a light beam which shines onto the bottom surface of the paw, through the glass floor of the testing enclosure. The device is electronically controlled and automated, and measurements are made of the number of seconds that elapse after the light beam begins to shine on the bottom of the paw, until the rat lifts its paw.

To ensure that the surgical operation itself did not affect the outcomes, comparative tests using each dosage level were also carried out on control populations of “sham-operated” animals. These animals were subjected to the same type of anesthesia and incisions on one limb, but no loop of suture material was placed around the sciatic nerve.

In all animals, the other hind limb was not operated on, and provided an additional control, generated by the same animal.

The results of these control experiments (not shown) demonstrated that sham-operated limbs in control animals, and unoperated limbs in test animals, responded identically to the thermal stimulus, under all treatment conditions.

To establish baseline values for pain sensitivity of each limb in each group of animals, each limb was tested initially for thermal pain sensitivity before the ligation operation was performed (pre-surgery). After 8 days, each limb was again tested before any drug treatment was administered (shown by the “pre-treatment” legend in all graphs). After drug administration, each limb was tested for thermal pain sensitivity at hourly intervals, for 4 hours.

Saline controls and all drugs were administered by intraperitoneal (IP) injection, i.e., hypodermic injection into the abdominal region. This is a convenient and reliable method of drug administration, for tests involving rats.

The test results are shown graphically in the Figures; each circle indicates the mean value, and the vertical bars show the standard error of the mean (SEM). In these graphs, the vertical position of each open circle indicates how many seconds elapsed (on average) between the commencement of the light beam which warmed the lower surface of the foot, and the act of lifting the hyper-sensitized hindpaw having the ligated nerve; by comparison, the vertical position of each darkened circle indicates how many seconds elapsed (on average) between the beginning of the thermal stimulus and lifting of the hind paw of the unoperated limb. Accordingly, a large vertical separation between a light circle (nerve-ligated paw) and a dark circle (control paw) indicates that the test animals were not well protected against neuropathic pain by the drug(s) used, at that dosage. By contrast, closely aligned dark and light circles indicate that the test animals were effectively protected against neuropathic pain by the drug(s) at the indicated dosage(s), at that point in time.

Large vertical gaps, showing a lack of effective pain control, are clearly indicated by the use of saline controls to test nerve-ligated animals, as shown in FIG. 1. Failure of the open circles to move vertically upward at any post-treatment interval, in a manner which reduced or closed the gap between the open circles and the dark circles, indicates that the inactive saline treatment did not provide any protection against neuropathic pain, in the nerve-ligated limb.

### Example 2

Procyclidine Alone

The graphs in FIG. 2 show that IP injection of procyclidine alone, at 10 mg/kg (FIG. 2A) or 25 mg/kg (FIG. 2B) did not significantly change the pain sensitivity in either limb, as evidenced by the fact that both the open circles and dark circles remain at their pre-treatment vertical levels at each post-treatment interval. At all times, the gap between the open and dark circles remained widely separated.

The graph in FIG. 2C shows that IP injection of procyclidine at 50 mg/kg significantly changed the level of pain sensitivity in the ligated limb, as indicated by a closing of the gap between the open and dark circles, at 1 hour post-treatment. Under this treatment condition, the rat shows a longer delay in withdrawing the paw of the ligated limb, so that paw withdrawal latency became essentially the same for each limb.

Complete closing of the gap (i.e., complete relief from neuropathic pain) occured at only the 1 hour post-treatment interval; by the second set of tests, at 2 hours post-treatment, the extent of pain relief had dropped off significantly.

At this dose of procyclidine, the rats did not show obvious signs of discomfort or detectable sensorimotor disturbances; however, a comparable dose in humans would very likely cause substantial discomfort (dry mouth, blurred vision, gastrointestinal disturbances) due to the strong anticholinergic action of procyclidine.

### Example 3

Clonidine Alone

Clonidine also was tested at several dosages, by itself, using the same procedures described in Example 1. The graphs in FIGS. 3A, 3B and 3C show the pain sensitivity in each limb when the treatment used was IP injection of clonidine at 0.025 mg/kg, 0.05 mg/kg or 0.075 mg/kg, respectively.

At the two lower doses (0.025 mg/kg and 0.05 mg/kg), clonidine showed no significant relief of neuropathic pain.

At the highest dose tested (0.075 mg/kg), significant and relatively sustained neuropathic pain relief was shown. However, at that dosage, the rats displayed significant sedation. In humans, a comparable dose would very likely produce both severe sedation, and a lowering of blood pressure to an unacceptable and potentially dangerous degree.

### Example 4

Combination of Procyclidine and Clonidine

Using the same testing procedures described above, the effects of co-administering clonidine at a dose of 0.025 mg/kg together with procyclidine at either a dose of 10 mg/kg (FIG. 4A) or 25 mg/kg (FIG. 4B) were evaluated.

Both of these graphs show that when procyclidine and clonidine were administered together, the combination was effective in providing sustained relief from neuropathic pain.

It should be noted that, in both sets of tests, each drug was administered at a dose that, by itself, did not offer any substantial pain relieving effects. The 10 mg/kg dosage of procyclidine corresponds to FIG. 2A, which clearly shows that at that dosage, procyclidine offered no protection whatever against neuropathic pain. The 0.025 mg/kg dosage of clonidine corresponds to FIG. 3A, was shows that, at that dosage, clonidine by itself was totally ineffective in providing any protection against neuropathic pain. The 25 mg/kg dosage of procyclidine corresponds to FIG. 2B, which shows that at that dosage, procyclidine offered only short-term and incomplete protection against neuropathic pain.

### Example 5

Reflex Testing to Confirm Hindpaw Control

In the experiments described in Examples 1-4, above, eight days after the rats had undergone surgical placement of a ligature around the sciatic nerve, and before they were subjected to drug treatment or a thermal pain stimulus, each animal was tested for the presence of certain types of reflexes that must be intact in order for the rat to promptly lift its paw in response to a stimulus.

One such evaluation is performed by holding the animal by its torso, with its lower limbs hanging downward, and bringing the top surface of a hind paw in contact with the edge of a table top. All animals with an intact placing reflex will immediately lift its hindpaw and place it on top of the table, in response to this stimulus. Performance of this test ensures that the ligature around the sciatic nerve has not impaired the animal's ability to lift its paw. If any animal was unable to perform that function without impairment, it was removed from further testing.

### Example 6

Additional Tests to Evaluate Adverse Side Effects

Studies were performed on separate groups of rats (not used in the pain testing sessions), to determine whether drug treatments at the doses administered in Example 2-4 would produce detectable impairments to reflex response, motor control, or behavior. These studies helped clarify whether the drug dosages that proved successful in relieving neuropathic pain might produce adverse side effects that would preclude using this drug combination in humans for the relief of chronic pain.

To address these issues, rats with sciatic nerve ligations, and rats with only sham sciatic nerve ligations, were treated with various doses of procyclidine, clonidine, or the two agents together. They were then evaluated, using the following battery of sensorimotor and activity tests:

Sensorimotor Battery: Testing was done at 1 hour post-treatment, which corresponded with the time of peak effect of drugs on paw withdrawal latency. Briefly, the sensorimotor battery consisted of five tests (Wozniak et al 1990) that are known to be able to detect significant muscle weakness, incoordination, somnolence, or other behavioral impairments.

For all of the tests in this sensorimotor battery, all treatment groups first received a habituation trial, to allow each rat to acclimate to the test situation. In all tests, protective padding was placed under any elevated apparatuses, to prevent pain or injury from falling. The tests performed were as follows:

(1) Plank test. A rat was timed for how long it could remain on a wooden plank that is 3 cm wide, elevated 61 cm above the floor.

(2) Walking initiation (also a test of sedation and activity level). Each rat was placed in the middle of a square outlined by white cloth tape (50×50 cm) on a smooth black surface of a large table top, and was timed for how long was required for the rat to leave the square (place all four paws outside the square).

(3) Platform. Each rat was timed for how long it could remain perched on an elevated (61 cm above the floor) platform of small dimensions (7.6×15.2 cm).

(4) Inclined screen. Each rat was placed in the middle of an elevated (61 cm above the floor) wire mesh grid (8 squares per 10 cm) which was inclined at 60 degrees with the floor. The rat's head was oriented downward toward the floor, and it was timed for how long it could continue to hold onto the screen.

(5) One hour activity level. Locomotor activity was evaluated beginning at 0.5 hr following drug treatment, and lasting for 1 hr. In this test, the rat was placed in a cage equipped with three pairs of photoelectric cells, placed at regular intervals across the width of the cage, so that the animal's activity was monitored by automatically recording the number of “beam breaks” registered as the animal moved about the cage.

The results indicated that clonidine, administered by itself at a dose of 0.050 mg/kg, did reduce activity levels, but only slightly and to a barely significant degree. At a dose of 0.075 mg/kg, clonidine substantially reduced activity levels, indicating that at that does, it had substantial sedating effects.

None of the procyclidine or combined procyclidine-plus-clonidine treatment regimens that proved effective for relieving neuropathic pain was associated with any impaired performance on any of the above described sensorimotor tests.

Thus, there has been shown and described a new and useful combined drug treatment for reducing neuropathic and/or other chronic pain. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

References

Aaronsen, L. M., et al, “Nociceptive action of excitatory amino acids in the mouse: effects of spinally administered opioids, phencyclidine and sigma agonists,” J Pharmacol Exp Ther 243: 9-19 (1987)

Abbott, N. J., et al, “Transporting therapeutics across the blood-brain barrier,” Mol Med Today 2: 106-13 (1996)

AMA Drug Evaluations, First Edition, 1971, American Medical Association, Chicago, Ill.

Aprison, M. H., et al, “Comparison of binding mechanisms at cholinergic, serotonergic, glycinergic and GABAergic receptors,” J Neurosci Res. 43: 127-36 (1996)

Backonja, M., et al, “Response of chronic neuropathic pain syndromes to ketamine: a preliminary study,” Pain 56: 51-57 (1994)

Begley, D. J., “The blood-brain barrier: principles for targeting peptides and drugs to the central nervous system,” J Pharm Pharmacol 48: 136-46 (1996)

Bennett, G. J., et al, “A peripheral mononeuropathy in rat produces disorders of pain sensation like those seen in man,” Pain 33: 87-107 (1988)

Bonate, P. L., “Animal models for studying transport across the blood-brain barrier,” J Neurosci Methods 56: 1-15 (1995)

Brightman, M. W., et al, “nonpermeable and permeable vessels of the brain,” NIDA Research Monographs 120: 87-107 (1992)

Buerkle, H., et al, “Opioidergic and cholinergic peripheral pain mechanisms,” Acta Anaesthesiol Scand Suppl 111: 184-6 (1997)

Bylund, D. B., “Pharmacological characteristics of alpha-2 adrenergic receptor subtypes,” Ann N Y Acad Sci 763: 1-7 (1995)

Choi, D. W., “Excitotoxic cell death,” J Neurobiol 23: 1261-1276 (1992)

Cotecchia, S., “The alpha 1a and alpha 1b-adrenergic receptor subtypes: molecular mechanisms of receptor activation and of drug action,” Pharm Acta Helv 74: 173-9 (2000)

Davar, G., et al, “MK-801 blocks the development of thermal hyperalgesia in a rat model of experimental painful neuropathy,” Brain Res 553: 327-330 (1991)

DeBoer, A. G., et al, “The transference of results between blood-brain barrier cell culture systems,” Eur J Pharm Sci 8: 1-4 (1999)

Doze, V. A., et al, “Dexmedetomidine produces a hypnotic-anesthetic action in rats via activation of central alpha-2 adrenoceptors,” Anesthesiology 71: 75-79 (1989)

Eisenach, J. C., et al, “Alpha-2 adrenergic agonists for regional anesthesia: A clinical review of clonidine,” Anesthesiology 85: 655-74 (1996)

Everitt, B. J., et al, “Central cholinergic systems and cognition,” Annu Rev Psychol 48: 649-84 (1997)

Farber, N. B., et al, “α2 adrenergic agonists prevent MK-801 neurotoxicity,” Neuropsychopharmacology 12: 347-349 (1995)

Frishman, W. H., et al, “Alpha-adrenergic blocking drugs in clinical medicine,” J Clin Pharmacol 39: 7-16 (1999)

Geroldi, C., et al, “Manipulation of the cholinergic system,” Funct Neurol. 12: 187-91 (1997)

Goodman and Gilman, eds., The Pharmacological Basis of Therapeutics, fifth edition (Macmillan, 1975)

Goodman and Gilman, eds., The Pharmacological Basis of Therapeutics, eighth edition (Pergamon Press, 1990)

Green, J. J., et al, “Transdermal clonidine therapy and nicotine withdrawal,” West J Med 151: 79-80 (1989)

Handa, F., et al, “Effects of oral clonidine premedication on side effects of intravenous ketamine anesthesia,” J Clin Anesth 12: 19-24 (2000)

Israel, M., et al, “Acetylcholine release and the cholinergic genomic locus,” Mol Neurobiol 16: 1-20 (1998)

Iversen, S. D., “Behavioural evaluation of cholinergic drugs,” Life Sci. 60: 1145-52 (1997)

Jevtovic-Todorovic, V., et al, “Clonidine potentiates the neuropathic pain relieving action of MK-801 while preventing its neurotoxic and hyperactivity side effects,” Brain Res 781: 202-211 (1998)

Kamibayashi, T., et al, “Alpha-2 adrenergic agonists,” Can J Anaesth. 44: R13-22 (1997)

Kamibayashi T, et al, “Clinical uses of alpha2-adrenergic agonists,” Anesthesiology 93: 1345-9 (2000)

Kniesel, U., et al, “Tight junctions of the blood-brain barrier,” Cell Mol Neurobiol 20: 57-76 (2000)

Kristensen, et al, “The NMDA-receptor antagonist CPP abolishes neurogenic ‘wind-up pain’ after intrathecal administration in humans, Pain 51: 249-253 (1992)

Kroll, R. A., et al, “Outwitting the blood-brain barrier for therapeutic purposes: osmotic opening and other means,” Neurosurgery 42: 1083-99 (1998)

Kubo, T., “Cholinergic mechanism and blood pressure regulation in the central nervous system,” Brain Res Bull 46: 475-81 (1998)

Langer, R., et al, “New directions in CNS drug delivery,” Neurobiol Aging 10: 642-4 (1989)

Levanen, J., “Dexmedetomidine premedication attenuates ketamine-induced cardiostimulatory effects and postanesthetic delirium,” Anesthesiology 82: 1117-25 (1995)

Liggett S B, “Pharmacogenetics of beta-1- and beta-2-adrenergic receptors,” Pharmacology 61: 167-73 (2000)

Louis, W. J. et al, “Sites of action of alpha-2 agonists in the brain and periphery,” Am. J. Cardiol. 61: 15D-17D (1988)

Mao, J., et al, “Intrathecal MK-801 and local nerve anesthesia synergistically reduce nociceptive behaviors in rats with experimental peripheral mononeuropathy,” Brain Res 576: 254-262 (1992)

Moens, Y., et al, “A comparative study of medetomidine/ketamine and xylazine/ketamine anesthesia in dogs,” The Veterinary Record 127: 567-571 (1990)

Nevalainen, T., et al, “Evaluation of anesthetic potency of medetomidine-ketamine combination in rats, guinea pigs, and rabbits,” Acta Veterinaria Scandinavica 85: 139 (1989)

Newcorn, J. H., et al, “Alpha 2 adrenergic agonists: neurochemistry, efficacy, and clinical guidelines for use in children,” Pediatr Clin North Am 45: 1099-22 (1998)

Oda, Y., “The distribution of cholinergic neurons in the human central nervous system,” Histol Histopathol 15: 825-34 (2000)

Olney, J. W., “Brain lesions, obesity, and other disturbances in mice treated with monosodium glutamate,” Science 164: 719-721 (1969)

Olney, J. W., et al, “Anti-Parkinsonian agents are phencyclidine agonists and N-methyl aspartate antagonists,” Eur. J. Phamacol. 142: 319-320 (1987)

Olney, J. W., “Excitotoxic amino acids and neuropsychiatric disorders,” pp. 47-71 in Annual Review of Pharmacology and Toxicology, Volume 30 (R. George et al, eds., Annu Rev Inc, Palo Alto, Calif. (1990)

Olney, J. W., et al, “NMDA antagonist neurotoxicity: Mechanism and protection,” Science 254: 1515-1518 (1991)

Pardridge, W. M., “CNS drug design based on principles of blood-brain barrier transport,” J Neurochem 70: 1781-92 (1998)

Pardridge, W. M., “Blood-brain barrier biology and methodology,” J Neurovirol 5: 556-69 (1999)

Patocka, J., “Central cholinergic nervous system and cholinergic agents,” Acta Medica (Hradec Kralove) 41: 99-108 (1998)

Physicians Desk Reference, 47th edition, Medical Economics Data, Montvale, N.J. (1993)

Poyhia, R., “Cholinergic mechanisms of analgesia,” Acta Anesthesiol Scand 44: 1033-1034 (2000)

Raigorodsky, G., et al, “Intrathecal NMDA activates both nociceptive and antinociceptive systems,” Brain Res 422: 158-62 (1987)

Rochat, B., et al, “Drug disposition and targeting: Transport across the blood-brain barrier,” Pharm Biotechnol 12: 181-200 (1999)

Rosin, D. L., “Distribution of alpha 2A- and alpha 2C-adrenergic receptor immunoreactivity in the central nervous system,” Methods Mol Biol 126: 475-505 (2000)

Rubin, L. L., “The cell biology of the blood-brain barrier,” Annu Rev Neurosci 22: 11-28 (1999)

Ruffolo, R. R., et al, “Pharmacologic and therapeutic applications of α2-adrenoceptor subtypes,” Annu Rev Pharmacol Toxicol 32: 243-279 (1993)

Saunders, C., et al, “Localization and trafficking of alpha2-adrenergic receptor subtypes in cells and tissues,” Pharmacol Ther. 84: 193-20 (1999)

Segal, M., “Overview of drugs used in treating drug-induced dependence,” Int J Addict 20: 1693-1706 (1985)

Seltzer, Z., et al, “Modulation of neuropathic pain behavior in rats by spinal disinhibition and NMDA receptor blockade of injury discharge,” Pain 45: 69-75 (1991)

Siegel, E. G., et al, “Investigation of clonidine and lofexidine for the treatment of barbiturate withdrawal in mice,” Vet Hum Toxicol 27: 503-5 (1985)

Stornetta, R. L., et al, “Alpha-2 adrenergic receptors: Immunohistochemical localization and role . . . ” Ann N Y Acad Sci. 763: 541-51 (1995)

Strosberg, A. D., “Structure, function, and regulation of the three beta-adrenergic receptors,” Obes Res 3 Suppl 4: 501S-505S (1995)

Sullivan, J. P., et al, “Cholinergic channel activators: novel opportunities for the treatment of CNS disorders,” Proc West Pharmacol Soc 38: 127-30 (1995)

Townsend, G. C., et al, “In vitro models of the blood-brain barrier to study bacterial meningitis,” Trends Microbiol 3: 441-5 (1995)

Tune, L. E., et al, “New cholinergic therapies: treatment tools for the psychiatrist,” J Clin Psychiatry 59 Suppl 13: 31-5 (1998)

Verstegen, J., et al, “Medetomidine/ketamine anaesthesia in cats,” Acta Veterinaria Scandinavica 85: 117-123 (1989)

Washton, A. M., et al, “Opiate withdrawal using lofexidine, a clonidine analogue with fewer side effects,” J Clin Psychiatry 44: 335-337 (1983)

Weinstock, M., “Possible role of the cholinergic system and disease models,” J Neural Transm Suppl 49: 93-102 (1997)

Woolf, C. J., “Recent advances in the pathophysiology of acute pain,” Br J Anaesth 63: 139-146 (1989)

Wozniak, D. F., “Behavioral effects of MK-801 in the rat,” Psychopharmacology 101: 47-56 (1990)

Yamamoto, T., et al, “Spinal pharmacology of thermal hyperesthesia induced by constriction injury of sciatic nerve: Excitatory amino acid antagonists,” Pain 49: 121-128 (1992)

Zimmermann, H., et al, “Molecular properties and cellular distribution of cholinergic synaptic proteins,” Prog Brain Res 109: 31-40 (1996)

1. A method for treating chronic pain, comprising the step of administering, to a mammal suffering from chronic pain, a drug combination comprising:
(a) a first drug which is an aryl-cyclo-alkanolamine compound or a prodrug, salt, isomer, analog, or derivative thereof, and which is pharmaceutically acceptable, and which suppresses activity at NMDA-type glutamate receptors; and,
(b) a second drug which stimulates activity at alpha-2 adrenergic receptors,
wherein the first and second drugs are administered at dosages which, when combined, provide synergistic and therapeutically effective relief from chronic pain, wherein such relief lasts longer than comparable pain relief provided by either drug alone, and wherein the combination causes lower levels of adverse side effects than either drug administered by itself would cause at a dosage which provides comparable short-term relief from chronic pain.

2. The method according to claim 1 wherein the first drug is selected from the group consisting of procyclidine, biperiden, triperiden, trihexyphenydyl, glycopyrrolate, hexocylium, oxyphenonium, tridihexethyl, oxyphencyclomine, mepenzolate, piperodolate, isopropamide, thiphenamil, adiphenine, and dicyclomine, and prodrugs, salts, isomers, analogs and derivatives thereof which are pharmaceutically acceptable, and which suppresses activity at NMDA-type glutamate receptors.

3. The method according to claim 1 wherein the second drug is selected from the group consisting of clonidine, iodoclonidine, guanabenz, guanfacine, xylazine, lofexidine, medetomidine, dexmedetomidine, tizanidine, rilmenidine, azepexole, α-methyldopa, and α-methylnoradrenaline, and prodrugs, salts, isomers, analogs and derivatives thereof which are pharmacologically acceptable, and which stimulate activity at α2 adrenergic receptors.

4. The method according to claim 1 wherein each drug is administered on a daily basis, in a painless manner that does not require a hypodermic injection.

5. The method according to claim 4 wherein each drug is administered by means selected from the group consisting of oral ingestion and transmembrane permeation.

6. A pharmacological mixture suited for daily treatment of chronic pain, comprising a combination of:
(a) a first drug which is an aryl-cyclo-alkanolamine compound or a prodrug, salt, isomer, analog, or derivative thereof, and which is pharmaceutically acceptable, and which suppresses activity at NMDA-type glutamate receptors; and,
(b) a second drug which stimulates activity at α2 adrenergic receptors,
wherein the first and second drugs are present in the mixture at relative concentrations which, when combined, can be administered to a mammal suffering from chronic pain, in a manner which will provide chronic pain relief which lasts longer than comparable pain relief that can be provided by either drug alone, and wherein the combination causes lower levels of adverse side effects than either drug administered by itself would cause at a dosage which provides comparable short-term relief from chronic pain.

7. The pharmacological mixture of claim 6 wherein the first drug is selected from the group consisting of procyclidine, biperiden, triperiden, trihexyphenydyl, glycopyrrolate, hexocylium, oxyphenonium, tridihexethyl, oxyphencyclomine, mepenzolate, piperodolate, isopropamide, thiphenamil, adiphenine, and dicyclomine, and prodrugs, salts, isomers, analogs and derivatives thereof which are pharmaceutically acceptable, and which suppresses activity at NMDA-type glutamate receptors.

8. The method according to claim 6 wherein the second drug is selected from the group consisting of clonidine, iodoclonidine, guanabenz, guanfacine, xylazine, lofexidine, medetomidine, dexmedetomidine, tizanidine, rilmenidine, azepexole, α-methyldopa, and α-methylnoradrenaline, and salts, isomers, analogs and derivatives thereof which are pharmacologically acceptable, and which stimulate activity at α2 adrenergic receptors.

9. The pharmacological mixture of claim 6 wherein the first drug and the second drug are both present in a unitary dosage form.

10. The pharmacological mixture of claim 9 wherein the unitary dosage form is designed for oral ingestion.

11. The pharmacological mixture of claim 10 wherein the unitary dosage form is selected from the group consisting of tablets and capsules.

12. The pharmacological mixture of claim 9 wherein the unitary dosage form comprises a skin patch.

13. The pharmacological mixture of claim 9, wherein the unitary dosage form is provided by controlled concentrations of the first drug and the second drug in a nondivided material selected from the group consisting of liquids, aerosols, and powders.

14. The pharmacological mixture of claim 13, wherein the nondivided material is packaged in a dispensing device that is capable of dispensing a predetermined volume of the nondivided material each time the device is used.

15. A pharmacological article of manufacture comprising a unitary dosage formulation which contains:
(a) a first drug which is an aryl-cyclo-alkanolamine compound or a prodrug, salt, isomer, analog, or derivative thereof, and which suppresses activity at NMDA-type glutamate receptors; and,
(b) a second drug which stimulates activity at alpha-2 adrenergic receptors,
wherein the first and second drugs are combined with each other in the unitary dosage formulation in dosages which, when combined, provide synergistic and therapeutically effective relief from chronic pain, wherein such relief lasts longer than comparable pain relief provided by either drug alone, and wherein the combination causes lower levels of adverse side effects than either drug administered by itself would cause at a dosage which provides comparable short-term relief from chronic pain.

16. The pharmacological article of manufacture of claim 15 wherein the first drug and the second drug are both present in a unitary dosage form.

17. The pharmacological mixture of claim 16 wherein the unitary dosage form is selected from the group consisting of tablets and capsules.

18. The pharmacological article of manufacture of claim 16 wherein the unitary dosage formulation comprises a skin patch.

19. The pharmacological article of manufacture of claim 16 wherein the unitary dosage form is provided by controlled concentrations of the first drug and the second drug in a nondivided material selected from the group consisting of liquids, aerosols, and powders.

20. The pharmacological mixture of claim 19, wherein the nondivided material is packaged in a dispensing device that is capable of dispensing a predetermined volume of the nondivided material each time the device is used.
